(19) United States
(12) Patent Application Publication
(10) Pub. No.: US 2010/0273738 A1
(43) Pub. Date: Oct. 28, 2010

(54) INTEGRATED PATIENT MANAGEMENT AND CONTROL SYSTEM FOR MEDICATION DELIVERY

(76) Inventors: CHRISTIAN P. VALCKE, Orlinda, CA (US); Michael Gorin, Los Altos, CA (US); James E. Gharib, San Diego, CA (US)

Correspondence Address: O''Melveny & Myers LLP;IP&T Calendar Department LA-13-A7, 400 South Hope Street, Los Angeles, CA, 90071-2899, US

(21) Appl. No.: 12/765,678

(22) Filed: Apr. 22, 2010

Related U.S. Application Data
(60) Provisional application No. 61/171,904, filed on Apr. 23, 2009.
(60) Provisional application No. 61/172,433, filed on Apr. 24, 2009.

(51) Int. Cl. C12Q 1/56; G01N 33/00; A61K 31/727

(52) U.S. Cl. 514/56; 422/68.1; 422/63; 435/13

(57) ABSTRACT

An integrated patient monitoring and control system is provided which includes a closed-loop control system for monitoring and adjusting the heparin infusion rate for a patient. The system includes a processor which uses a dynamic patient model that is continuously adjusted based on the patient's aPTT measurements to calculate an optimal heparin infusion rate to achieve an operator-input aPTT target range. The processor also includes a forecasting model to calculate the optimum sample time interval for measuring the patient's aPTT to calculate a new infusion rate. An automated sampling system, which includes a storage device for storing a series of assay devices, an advancement mechanism for moving the assay devices to a sample area, and a measurement device for analyzing a sample dispensed on the assay, is provided. The sampling system is used to repeatedly measure the patient's aPTT according to the sample time interval determined by the processor.

### RELATED APPLICATION INFORMATION

This application claims priority to and benefit of U.S. Provisional Application Ser. No. 61/171,904, filed Apr. 23, 2009, entitled “Automated Assay System for Closed-Loop Drug Delivery” and U.S. Provisional Application Ser. No. 61/172,433, filed Apr. 24, 2009, entitled “Systems and Apparatus for Automatic Closed-loop Heparin Delivery,” the content of which is incorporated by reference herein in their entirety as if fully set forth herein.

This application is related to U.S. Provisional Application Ser. No. 61/086,383, filed Aug. 5, 2008 (our Reference 037,028-002); U.S. Utility application Ser. No. 12/534,447, filed Aug. 3, 2009 (our Reference 037,028-006); U.S. Provisional Application Ser. No. 61/139,826, filed Dec. 12, 2008 (Our Reference 037,028-003); and U.S. Utility application Ser. No. 12/643,398, filed Dec. 11, 2009 (Our Reference 037,028-008), each of which are incorporated herein by reference in their entirety as if fully set forth herein.

### FIELD OF THE INVENTION

The invention relates generally to an automated closed-loop (feedback controlled) drug delivery system using an optimal sampling method and control system. More particularly, the invention relates to methods and apparatus for use in the administration of drugs, such as heparin as an anti-coagulant medicine used in the treatment of cardiovascular and neurovascular disease as well as deep-vein thrombosis and pulmonary embolic disease.

### BACKGROUND OF THE INVENTION

Millions of patients are treated with unfractionated heparin (UFH) in the acute care hospital setting to control their level of anticoagulation. These patients are monitored by a multi-step, labor intensive process to maintain their level of anticoagulation. This complex process leads to frequent human error, thus only 35%-50% of patients are within a safe range of heparin at any given time. The consequences of both under- and over-anticoagulation include death, heart attack, stroke, moderate to severe blood loss, tremendous strain on the patient and their loved ones, and millions of dollars in avoidable health care costs. The problem has become so serious that the Joint Commission recently issued a “Sentinel Event Alert”¹ regarding the prevention of errors related to heparin. Such alerts require immediate investigation and response for an event that carries a significant chance of a serious adverse outcome. Several approaches have been tried to improve control of heparin levels. These approaches include point-of-care monitoring and use of standardized nomograms. The attempts have yielded little if any improvement.

Heparin, alone or in conjunction with other antithrombotic agents, is the standard of treatment in patients with acute myocardial infarction (AMI), unstable angina (UA), thrombosis, deep vein thrombosis, or pulmonary embolism. Heparin produces a dose-dependent prolongation of the clotting process measured by the activated partial thromboplastin time (aPTT). However, the anticoagulant effects of heparin are variable. Previous studies have reported wide subject variation in the dose of heparin required to achieve and maintain a therapeutic aPTT². A study, published in February 2009 in Circulation,³ further confirmed that only 33% of patients receiving heparin had therapeutic anticoagulation. The consequences of too high or too low a level of anticoagulation can be serious.⁴ In patients with acute ischemic syndromes, inadequate anticoagulation may lead to recurrent thrombosis, and significant bleeding has occurred in patients at supra-therapeutic doses of heparin. When a fixed dose of heparin is used as conjunctive therapy to thrombolysis or in the treatment of AMI, a substantial percentage of patients can be above or below the aPTT therapeutic range at any point in time.

Heparin is a naturally-occurring anticoagulant that when administered intravenously prevents the formation of clots and extension of existing clots within the blood. It is used for a number of different conditions. It is given as a continuous infusion for management of acute coronary syndromes, stroke, pulmonary emboli and venous thrombosis. Since the goal of therapy is to achieve a target range of anticoagulation rapidly and then maintain that level for a period of time, continuous infusions are monitored periodically and the dose is adjusted. Heparin dosing can be complicated by a number of factors, including illness that it is being used to treat. Various factors, including disease state can affect heparin pharmacokinetics and pharmacodynamics. Thus monitoring and dose adjustment are required to optimize therapy primarily for anticoagulation for cardiovascular conditions, including acute coronary syndromes, myocardial infarction, atrial fibrillation, cardiopulmonary bypass surgery (CABG), percutaneous coronary intervention (PCI), deep vein thrombosis and pulmonary embolism.

In the administration of heparin, the objective is to achieve an activated partial thromboplastin time (aPTT) value that is 1.5× to 2× the patient's baseline aPTT. As a result of the difficulty in correctly titrating heparin dosage in any given patient, on average the desired aPTT range +/−15 seconds is achieved in only 30% to 40% of patients during the course of therapy.⁵

The worldwide market for unfractionated heparin is estimated at $400 million.⁶ The US market for unfractionated heparin is about $146 million. It is a generic drug with Baxter, APP and Hospira comprising 80% of the market.⁷ Sales of heparin have maintained a steady growth over the past few years. From June 2006 to June 2007, total US heparin sales units grew by 6%.⁸ With the recent Baxter heparin recall early in 2008, the market (unit sales) has declined slightly as a result of less supply available in the market; however with manufacturers such as APP increasing production capacity, heparin supply should recover within the year.

Heparin is associated with many medication errors as a result of its complex pharmacologic response and large inter-patient variability in response. According to the United States Pharmacopoeia (USP) MED-MARX⁹, during a five year period from 2003 to 2007, heparin medication errors totaled 17,000 out of more than 50,000 anticoagulation related medication errors.¹⁰ The majority of heparin errors occur during administration at the bedside (47.6%) followed by prescribing errors (14.1%), dispensing (13.9%) and transcribing and documenting (18.8%). A majority of these errors resulted from a failure to follow procedures and protocols.¹¹ These errors all result in significant economic costs to the health care system.

Close monitoring of patients on heparin is extremely important: too low a dose of heparin can lead to under anticoagulation while too high a dose can lead to serious bleeding. It is also important to bring patients into range as quickly as possible to avoid adverse outcomes. ¹² In studies of patients with acute coronary syndromes treated with intravenous heparin, increasing aPTT values were associated with increased bleeding episodes.¹³ At various times throughout therapy, only 50% of patients had aPTT values in the therapeutic range.¹⁴

Lower than required dosing levels of heparin can lead to episodes of thromboembolic complications in patients with acute coronary syndromes (ACS) or deep vein thrombosis while higher than required levels of heparin can lead to bleeding complications.¹⁵ In the recent “Can Rapid Risk Stratification of Unstable Angina Patients Suppress Adverse Outcomes with Early Implementation of the American College of Cardiology/American Heart Association Guideline (CRUSADE) initiative, it was observed that 49% of patients received excess dosing of unfractionated heparin leading to a significantly higher rate of major bleeding and need for transfusion as compared to patients who did not receive excess dosing.¹⁶

The problem has become so serious that the Joint Commission, which accredits all US hospitals issued a “Sentinel Event Alert”¹⁷ regarding the prevention of errors related to commonly used anticoagulants. Such alerts signal the need for immediate investigation and response for an event that carries a significant chance of a serious adverse outcome.

Current practices for the administration of heparin in an acute care setting involve many different steps and resources that can easily tax the hospital staff and lead to human error. General heparin dosing protocols (nomograms) may include the following steps: a standard initial bolus of heparin with a calculated infusion rate normally based on the patient's weight; instructions for drawing blood samples for partial thromboplastin time (aPTT) testing and orders for dosing adjustments in response to measured aPTT and optionally other values. The nurse will take a blood sample and send it to the central lab for analysis. The lab will provide the result to the nurse and the nurse will then evaluate the result and make the necessary adjustments to the dose based on the results. The nurse will check with the physician to verify dosing. Upon receiving approval from the physician, the nurse will make the necessary adjustment to the infusion rate. This process requires at least 1-2 hours to complete each time and is repeated every 4 to 6 hours over the course of approximately 2.5 days while the patient is receiving heparin.

As medication errors have continued to occur with heparin, sometimes causing serious complications, many hospitals and organizations have devised ways to try to minimize medication errors. Besides instituting nomograms for heparin administration, hospitals have tried other systems such as bar coding software that can identify and verify the drug and its concentration; inpatient anticoagulation services for heparin in which pharmacists run the services that provide daily pharmacy input on dosing and monitoring for patients on heparin; and automated medication dispensing systems.

The introduction of “smart” infusion pumps in the past few years have tried to address the issue of dosing errors before the patient suffers any negative effects. These smart pumps, which are still only used in approximately 50% of all hospitals in the US¹⁸, contain comprehensive drug libraries and standardized dosing units based on the specific acute care area of use. They also have dose calculators and alert systems if dosing falls out of pre-determined parameters or “guard-rails”. Nevertheless, recent reviews have concluded that many users of smart pumps bypass the safety features of the devices, and as a result medication errors continue to occur.¹⁹

Smart pumps attempt to prevent the nurse from inadvertently typing in a dose outside the standard dosing range. There is no provision for individualizing the dose for each patient, nor is there the ability to use a measure of patient response to adjust dosing. For medications with variable patient response (e.g. unfractionated heparin, insulin) the use of more individualized dosing and individualized adjustment according to a blood test has the potential to advance therapy and improve response.

Hospitals are increasingly concerned about medication errors. They are also in search of tighter control of critical parameters in the ICU, including anticoagulation and blood glucose. As a result, there is significant opportunity for a smart-controller that can integrate critical diagnostic assays and information to adjust patient dosing safely. With renewed focus on eliminating human error in drug administration of potent intravenous agents in the hospital, there is a large unmet need.

While previous systems have been described, see, e.g., Hillman et al., “Feedback Controlled Drug Delivery System”, U.S. Pat. No. 5,697,899, issued Dec. 16, 1997, Valcke et al., “Method and Apparatus For Closed-loop Drug Delivery”, U.S. Pat. No. 5,733,259, issued Mar. 31, 1998 and Gauthier et al., “Feedback Controlled Drug Delivery System”, U.S. Pat. No. 6,017,318, issued Jan. 25, 2000, all incorporated by reference herein in their entirety, they do not contain or integrate all of the advanced features in the current invention that are designed to further minimize medication errors and further improve the level of control.

Ordinarily, drug delivery systems are control systems having an input-output relationship. A drug input, such as an absolute amount or an infusion rate, produces a physiological response related to that input. Typically, the input (such as the infusion rate) is used to control some parameter associated with the response variable, such as desired anti-coagulation measurement.

Broadly speaking, drug delivery systems are either open-loop delivery systems or closed-loop delivery systems. An open loop delivery system is one in which the drug is delivered at a pre-determined rate without any direct or automatic adjustment in response to the physiological response variables and measurements. A closed-loop drug delivery system is one in which a drug is delivered in automatic response to feedback of a physical signal or measurement, which could include physiological variables or analytical measurements such as PT or aPTT. For closed-loop systems, we can also differentiate “near-patient” control where an operator provides the changes in infusion rate based on output generated from the control system and information that the operator has entered (e.g., patient characteristics, aPTT, value, etc.). Similarly, the control system can calculate the predicted response based on infusion rate information entered by the healthcare practitioner, even if different from the optimal rate. The control system can also provide information to the operator, on the optimal sampling times for aPTT to achieve the best control of heparin.

The input-output relationship is often described by a mathematical model and, except in very simplified circumstances, includes the concept of a dynamic system. In a dynamic system, the output behavior is a result not only of the current output but also of the history on previous inputs and the initial condition of the system. Furthermore, the input-output relationship can be a one-to-one (one input determines one output) or many-to one (many inputs affect many outputs) depending on the complexity of the system.

In a closed-loop delivery system, one must develop the control system in order to determine the optimal inputs to achieve a desired output for a dynamic system.

While numerous types of closed-loop systems exist, representative categories of control schemes include: linear-nonlinear, deterministic-stochastic, and adaptive-non-adaptive. For electro-mechanical systems, the behavior of the system may be well characterized and remains constant. In this case, the determination of optimal inputs can be often be calculated analytically and does not change during the course of the product use, for example a automotive cruise control system. In other systems, the knowledge of the input-output relationship may not be known or may change during the use of the application. In these cases, the representation of the dynamic system may be adjusted during the application as more information becomes available about the behavior of the input-output. This is known as an adaptive control system. For biological systems, there may be general, population based, information about input-output behavior. However, for each individual treatment, one may expect a range of distributions around the population based estimates and perhaps a change in response during the application. Mathematically, this may be represented by introducing a parameter set that contains one or more variables with a possible range of discrete or continuous values.

Closed-loop drug delivery systems have been used for therapeutic purposes to maintain a physiologic parameter. One specific example is the use of a closed-loop drug delivery system to control infusion of Nipride to control a patient's blood pressure. Such a system is described in Petre et al., “Infusion Pump Control”, U.S. Pat. No. 4,392,849. Such a system is designed to maintain stability of a physiological parameter, as opposed to variation of that parameter for diagnostic purposes. Yet further examples of closed-loop drug delivery systems for therapeutic purposes are disclosed in Newman, PCT Application WO 88/08729, entitled “Iontophoresis Drug Delivery System”, published Nov. 17, 1988. Various therapeutic closed-loop drug delivery applications are mentioned, including for medication delivery, control of blood pressure, insulin delivery and administration of pain killing drugs.

There are many unique and important obstacles presented in effective treatment utilizing a closed-loop drug delivery system, especially for the administration of heparin. For example, there is potentially a time delay for the effect of the administration on the systemic coagulation status, that being the time between the peripheral administration of the heparin and the physiological coagulation cascade. Second, it is well documented that different patients respond differently to a given drug amount, making response predictability more difficult. Third, as the disease condition or physiology of the patient changes, the response to the drug may change during the application of the drug. Fourth, safety monitoring of the drug response must be monitored and possible terminate drug delivery if condition persists. Finally, monitoring the response of the drug administration requires an analytical test on a blood sample requiring an intermittent sampling scheme since no continuous measurements of this physiological response are currently available.

### SUMMARY OF THE INVENTION

The invention relates generally to a closed-loop drug delivery system using an optimal sampling method and an adaptive control system for performing automated blood analysis, computing the optimal dosage and controlling a drug delivery system to administer the dose to a patient. More particularly, the invention relates to methods and apparatus for use in the administration of drugs, most particularly heparin as an anti-coagulant medicine in the treatment of cardiac disease. This apparatus and method can be used advantageously in the treatment of coronary artery disease by providing for feedback controlled drug delivery in a patient specific optimal treatment regimen. The description of the method can also be applied to a “near-patient” control setting where the operator changes the infusion rate based on calculated infusion rates based on input that they have provided (e.g., in the case of heparin, information on the value of aPTT, i.e., activated partial thromboplastin time).

In one embodiment, an integrated patient monitoring and control system is provided which includes a sampling infusion tubing set (SITS) also referred to as Blood Sampler Withdrawal Set (BSWS in FIGS. 1, 3, 5, and 6), the SITS being adapted for coupling to the patient to obtain a specimen from the patient, a sensor, the sensor being adapted to receive the specimen from the SITS and to analyze the sample, a medication control unit, the medication control unit receiving information from the sensor, and utilizing that information to determine medication dosing information specific to the patient, and a medication administration system, the medication administration system receiving the dosing information from the medication control unit, and adapted to cause administration of the medication to the patient. In one embodiment, the SITS is adapted for blood draw from the patient. Advantageously, the blood draw is performed in conjunction with a pneumatic pressure cuff, inflated so as to aid in blood draw.

In yet another embodiment, an automated blood sampling system, comprises a tourniquet, an indwelling catheter, a pressure measuring system, a pump, a disposable set, an optical source and detector, and a computer controlled adaptive algorithm. The system mechanizes blood draw by optimizing blood draw parameters such as by varying vacuum on the cuff, adjusting the rate of blood withdrawal, adjust pressure in the cuff, etc.

In another embodiment, a multi-parameter integrated patient monitoring and control system includes a sampling infusion tubing set (SITS), this set being adapted for coupling to the patient to obtain a specimen from the patient, a sensor, the sensor being adapted to receive the specimen from the SITS and to analyze the sample, the sensor including a first assay and at least a second assay, the assays testing for different medical conditions or different drugs, a medication control unit, the medication control unit receiving information from the sensor including information on the first and second assay, and utilizing that information to determine medication dosing information for the patient, and a medication administration system, the medication administration system receiving the dosing information from the medication control unit, the system including a first drug to be administered corresponding to the first assay and a second drug to be administered corresponding to the second assay, and adapted to cause administration of the medication to the patient. By way of example, the first assay could relate to blood clotting, e.g., aPTT, ACT, or Factor Xa value, and the first drug be heparin, and the second assay could relate to blood glucose level, and the second drug be insulin.

In yet another embodiment, a multi-parameter integrated patient monitoring and control system includes a SITS, the SITS being adapted for coupling to the patient to obtain a specimen from the patient, a sensor, the sensor being adapted to receive the specimen from the SITS and to analyze the sample, a medication control unit, the medication control unit receiving information from the sensor and at least one other patient information parameter, and utilizing that information to determine medication dosing information for the patient, and a medication administration system, the medication administration system receiving the dosing information from the medication control unit, and adapted to cause administration of the medication to the patient. In addition to the results of the first assay (that contains information relating to the patient response to the first drug being administered), a second item of patient information may be information from at least a second sensor or sensors or information relating to a first drug being administered, such as the drug level of the patient or information relating to the pharmacodynamic response of the patient to the first drug. The other patient information may also be the patient's vital signs, such as the blood pressure or heart rate of the patient, temperature and/or respiration rates.

In another embodiment, an integrated patient monitoring and control system is provided which includes a sample analysis system for intermittently determining the activated partial thromboplastin time (aPTT) or other coagulation assays in a fluid medium. The system preferably includes a series of assay devices, an aPTT measurement device, a storage device for storing a plurality of said assay devices individually hermetically sealed, and an automated mechanism, such as a motor, for repetitively advancing one of said assay devices to a sample application area to intermittently perform a diagnostic assay on a sample. In one implementation, the storage device comprises a cassette contain a series of assay devices, the assay devices preferably having been removed from original packaging, and being hermetically sealed by secondary packaging materials. The system operates under control of a control unit for exposing application site to a sample dispensing device, for example to load a blood or other fluid sample on the sample application area of the assaying device. Assaying is performed by a reader (e.g. optical), the sample being illuminated by a source of light, to measure an analyte in the fluid sample. A removal mechanism removes the assay device from the application area after completion of one diagnostic (aPTT) reading. Optionally, an integrated reservoir for collecting a liquid waste is provided. The process is repeated intermittently or continuously as directed by the drug delivery control system

In another embodiment, a control system and methods for automatic feedback control of delivery of a drug, such as heparin, are provided. The goal of a heparin control system specifically is to maintain the anticoagulation state of a patient within the prescribed safe limits. The system accomplishes this goal by calculating the appropriate infusion times and rates based on aPTT measurements that are made following each heparin infusion. The infusion rate calculated by the heparin control system is based on a pharmacodynamic (PD) model of heparin response. Based on measurements of patient response, the model parameters can be adjusted (using, for example, Bayesian estimation). In addition, a confidence interval that reflects the individual patient's variability in response to heparin infusions can also be assessed and constantly updated, either continuously or at periodic intervals, as part of the feedback loop. The goal of the system is to keep aPTT within the proscribed confidence limit for each patient. This constant updating function is a main contributor to the high quality of control that can be achieved.

In another embodiment, the drug delivery control system delivers heparin based on optimally sampled aPTT measurements to achieve a desired anticoagulation status of the patient. The overall drug delivery system consists of a hardware system and an expert, rule-based, control system. In one embodiment of a multi-parameter integrated patient monitoring and control system, the system, apparatus and methods all operate in an automatic feedback controlled manner to achieve drug delivery. Stated otherwise, the integrated patient monitoring and control system operates to monitor, sample, determine time and dosing requirements, and cause dosing without intervention by health care professionals (save for example a required response to an alert or alarm condition). In an alternate embodiment, the hardware can also be replaced in whole or in part by a user that provides, for example, the results (aPTT) at the times requested by the system, and manually adjusts the infusion rate in response to the expert-rule based control system. In this system the rule-based control system, the system is flexible enough to accept inputs (e.g., aPTT) at times other than that requested, and adapt to changes in infusion rate other than recommended by the system. In this mode, the system functions much like a GPS navigator for an automobile, where a driver makes a wrong turn, and it recalculates the route to get to the desired destination (albeit using taking a longer time to reach the destination).

In another embodiment, a method for monitoring and adjusting the infusion rate for delivering heparin to a patient is provided. The method generally comprises the following steps: obtaining a patient blood sample and measuring the patient's Activated Partial Thromboplastin Time (aPTT). The patient's aPTT measurement and a target aPTT range for the patient are input into a processor. The processor then calculates the optimal heparin infusion rate for the patient to achieve the target aPTT range. The processor includes a protocol based on a pharmacodynamic model of heparin response that is used to calculate the optimal infusion rate for the patient to achieve a target aPTT range. The pharmacodynamic model utilizes the patient's past history of infusion rates and responses as well as the current infusion rate and response, for example as indicated by the aPTT measurement response to calculate the optimal infusion rate. The processor also determines an optimal sample time interval for repeating the process to reassess the patient's aPTT measurement and adjust the heparin infusion rate to maintain the target aPTT range. In addition, the pharmacodynamic model is constantly adjusted using the patient's past history of heparin infusion rates and responses to tailor the model to the patient's individualized heparin response.

In one embodiment, the Integrated Patient Management and Control System uses a dynamic patient model to predict an aPTT response that is then used to calculate the optimum heparin infusion rate for the patient. The patient model takes into account the heparin response to the current infusion rate in calculating the optimum infusion rate for the patient. In addition, unlike previous systems, the dynamic patient model can also take into account the patient's history of responses to past infusion rates in calculating the optimal current infusion rate. Thus, each time the patient's response is measured, the patient model which will be used for making future adjustments to the infusion rate is also adjusted to reflect the additional data point. In some embodiments, the patient model is also used to predict the uncertainty in the aPTT response. This uncertainty can then be used to determine a confidence interval that reflects the individual patient's variability in response to heparin infusions. This confidence interval can be used to calculate the optimum sampling time, i.e., time interval between measurements of the patient's aPTT for monitoring and adjusting the heparin infusion rate.

In some embodiments, the drug delivery control system includes a software-based supervisor that issues alarms and alerts if certain preset conditions are detected. For example, the software based supervisor has the ability to notify the user if certain pre-set alarm conditions occur, such as, no sample input for an extend period, an unexpected patient response, input infusion rate that system expects will result in aPTT being out of range or the like. Preferably, alerts and alarms are sent to a central nursing station or to an assigned health care professional.

### BRIEF DESCRIPTION OF THE DRAWINGS

FIG. 1 shows the cycle of the sample withdrawal set, the sensor, the medication control unit and the drug delivery technology.

FIG. 2 is a schematic block diagram of the main components of a heparin control algorithm.

FIG. 3 is a detailed block diagram of the system.

FIG. 4 is a flowchart showing overall operation of the system.

FIG. 5 shows a perspective view of the integrated patient management and control system for medication delivery.

FIG. 6 shows a perspective view of an alternate embodiment of the integrated patient management and control system for medication delivery.

FIG. 7a shows a top down view of an assay showing alternating assay regions. FIG. 7b shows a top down view of an assay showing four differing assays.

FIG. 8 shows a front view of a representative display system.

FIG. 9 is a flowchart of the single cuff implementation of the system and methods.

FIG. 10 is a flowchart of the multi-cuff implementation of the system and methods.

FIG. 11 shows a loading mechanism and a sample application area in side view.

FIG. 12 shows a loading mechanism and a sample application area in top view.

FIG. 13 shows a slide tray arrangement of multiple test site locations in side view.

FIG. 14 shows a slide tray arrangement of multiple test site locations in side view.

FIG. 15 shows a planar carousel arrangement for multiple test site locations in top view.

FIG. 16 shows a planar carousel arrangement for multiple test site locations in side view.

FIG. 17 shows a fan fold arrangement of multiple test site locations in top view

FIG. 18 shows the fan fold arrangement of multiple test site locations in side view and a loading and delivery mechanism for the fan fold arrangement.

FIG. 19a is a flowchart of an embodiment of the control system illustrating the overall control system.

FIG. 19b is a flowchart of the pre-drug delivery phase of the embodiment of the control system illustrated in FIG. 19a.

FIG. 19c is a flowchart of the drug delivery phase of the embodiment of the control system illustrated in FIG. 19a.

### DETAILED DESCRIPTION

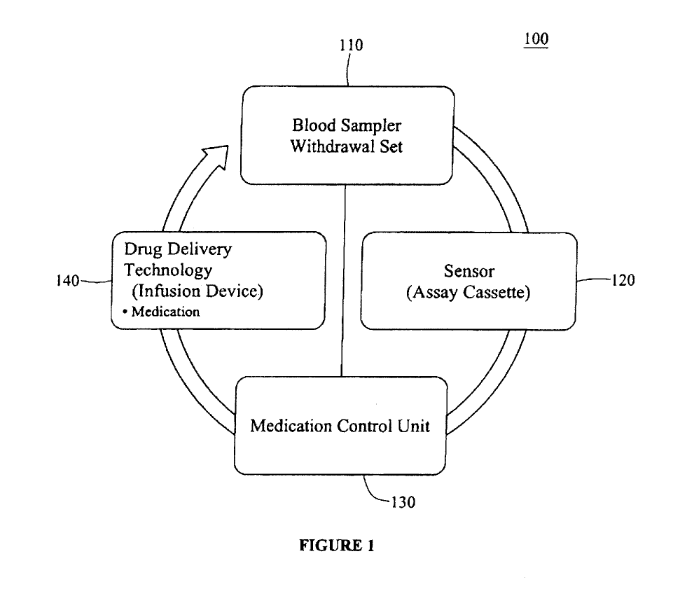

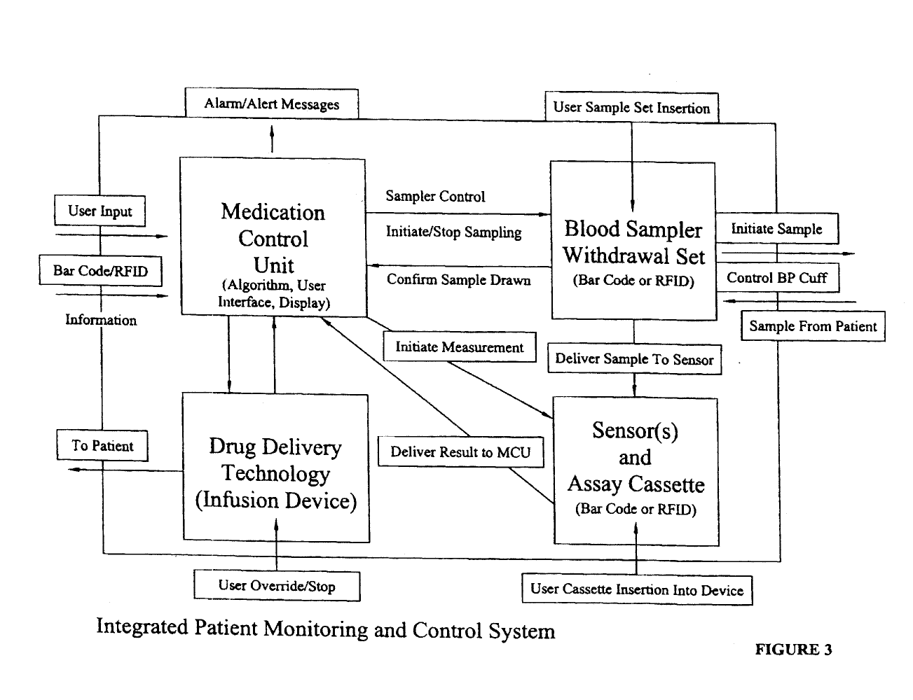

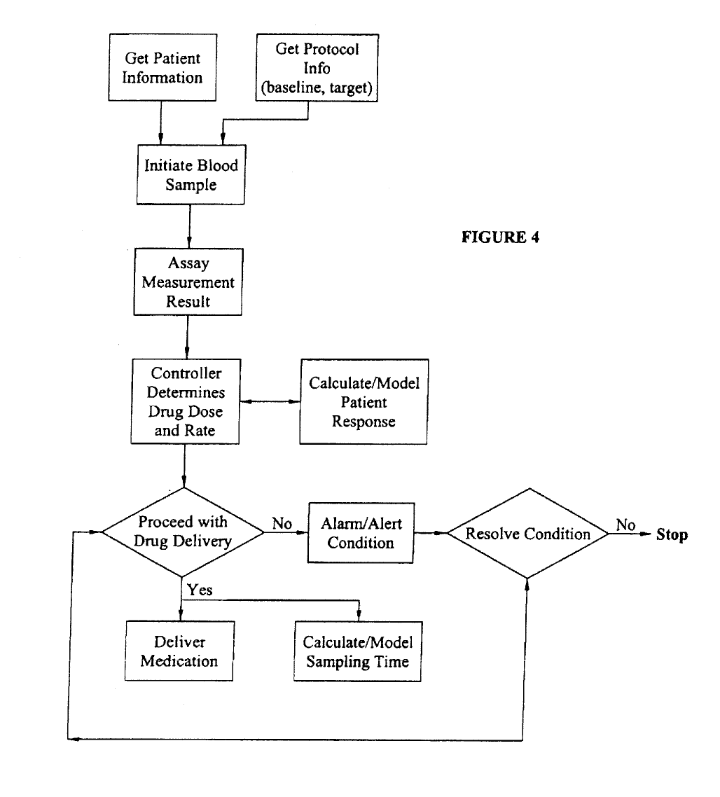

With particular reference to FIGS. 1, 2, 3, 4, 9 and 10, this invention describes an integrated patient measurement and control system 100 (IPMC) for delivering medications. The preferred elements of the system as depicted are the blood sampler/withdrawal tubing set (or SITS)—110, one or more sensors 120, a medication control unit 130 and an integrated drug delivery technology 140 through which medication can be delivered.

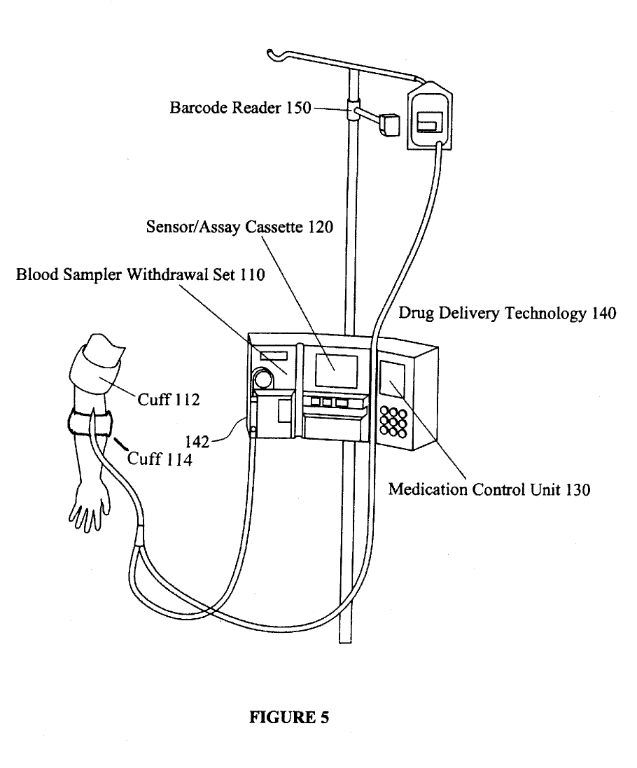

In one aspect, one of the key features of the IPMC System is an Integrated Drug Delivery Technology, shown in FIG. 5 is an integrated intravenous (IV) infusion pump. This integration minimizes the chance for communication errors that could occur with an external infusion device leading to potentially serious consequences such as infusion without proper feedback. Additional elements of the system include an integrated bar code reader (or RFID reader) 150 to read the name, dosage, and concentration of the medication to be delivered and patient ID to further minimize any medication delivery errors; intermittent sampling and control, and an inflatable tourniquet/constriction cuff that can be used in conjunction with the sampler device and medication control unit. The term cuff encompasses cuffs, including pneumatic cuffs, tourniquets or other forms of constriction devices. The system is capable of controlling different medications via interchangeable sensor and algorithms, or multiple medications through a multiplexed assay cassette.

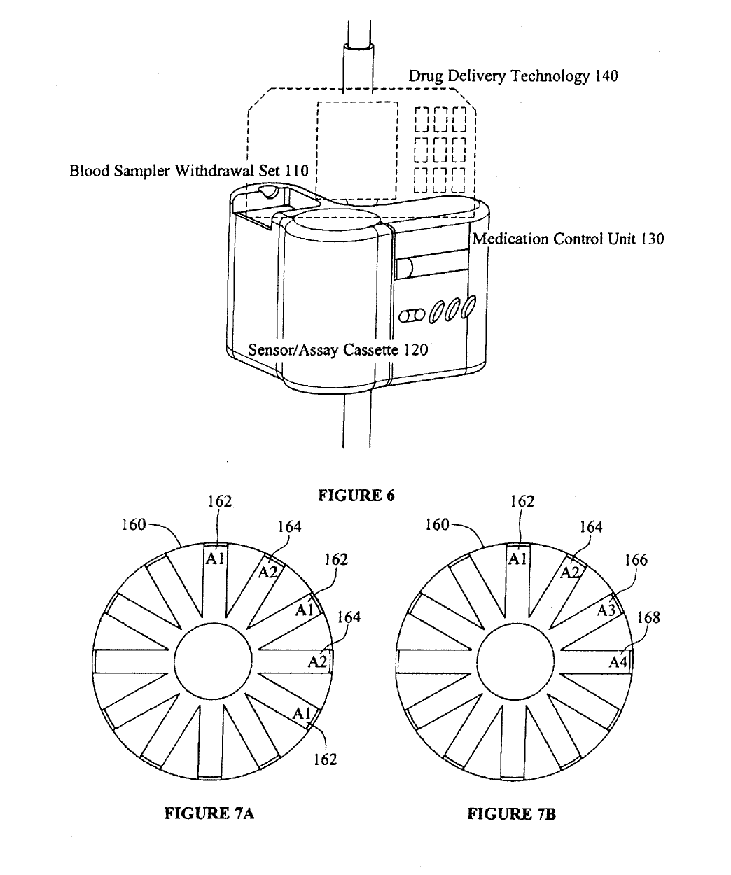

An alternative embodiment of the system is shown in FIG. 6, again containing integration of all of the elements described.

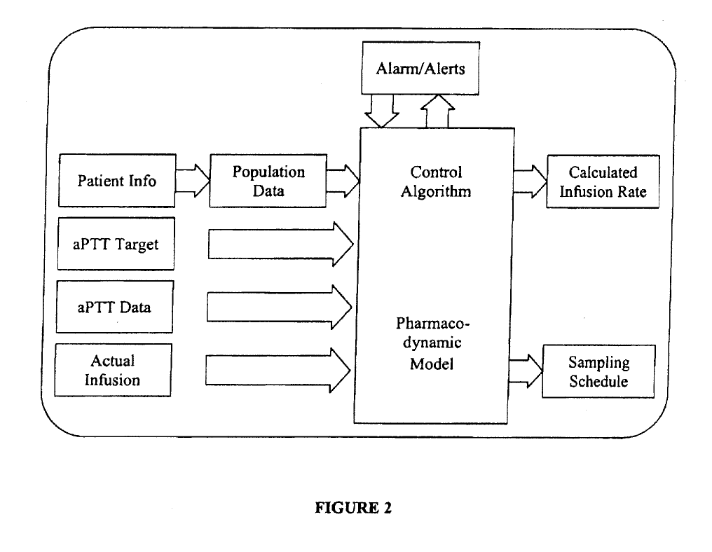

In another aspect, one of the key features of an integrated patient measurement and control system is an adaptive feedback control system. FIG. 2 shows the overall feedback control system as governed by a supervisory system that can monitor the input-output data for anomalies and trigger relevant signals to notify the operator (alerts) and if necessary stop the infusion (alarms). The control system is implemented in an electronic system, preferably a programmable system such as a microprocessor, microcontroller or embedded system.

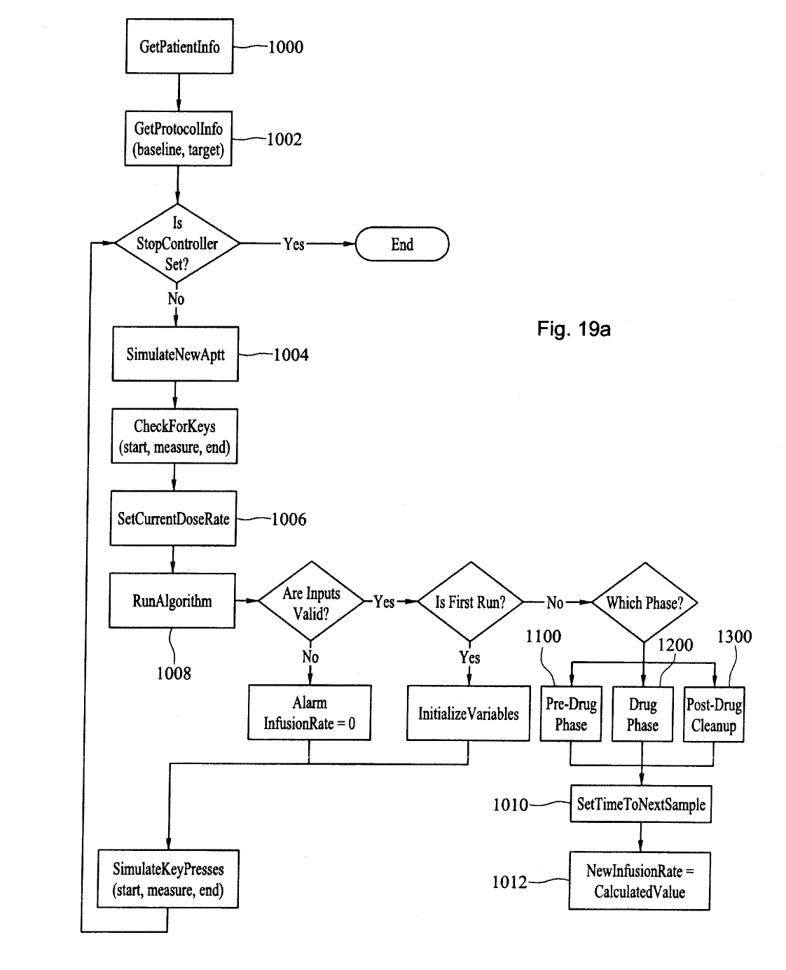

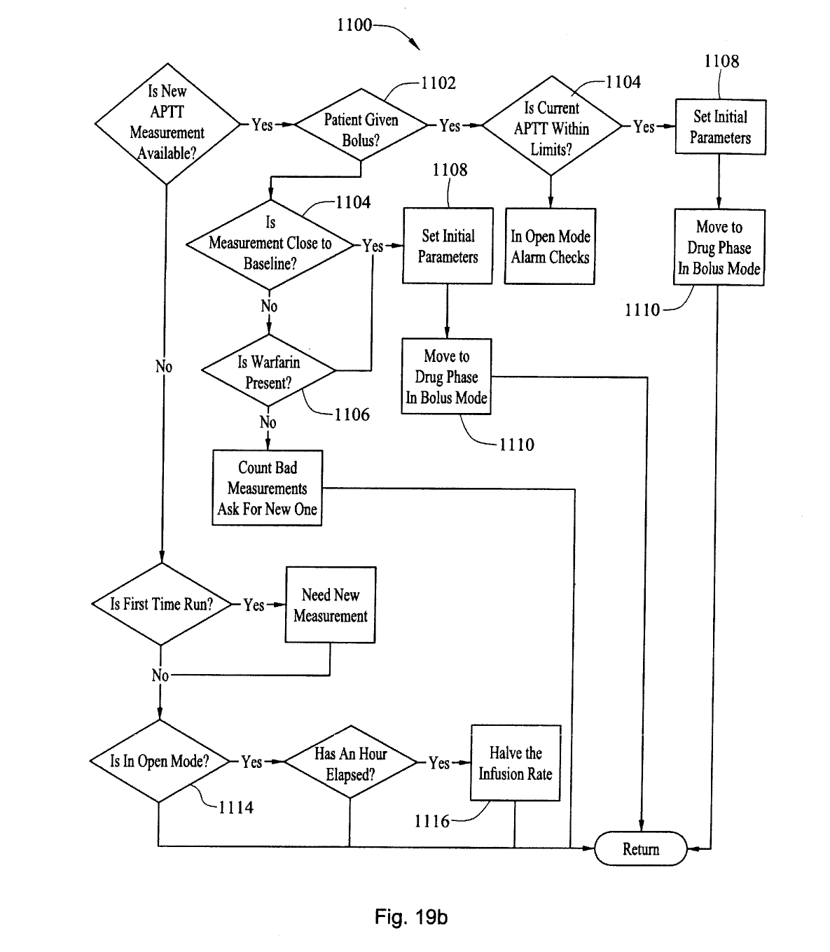

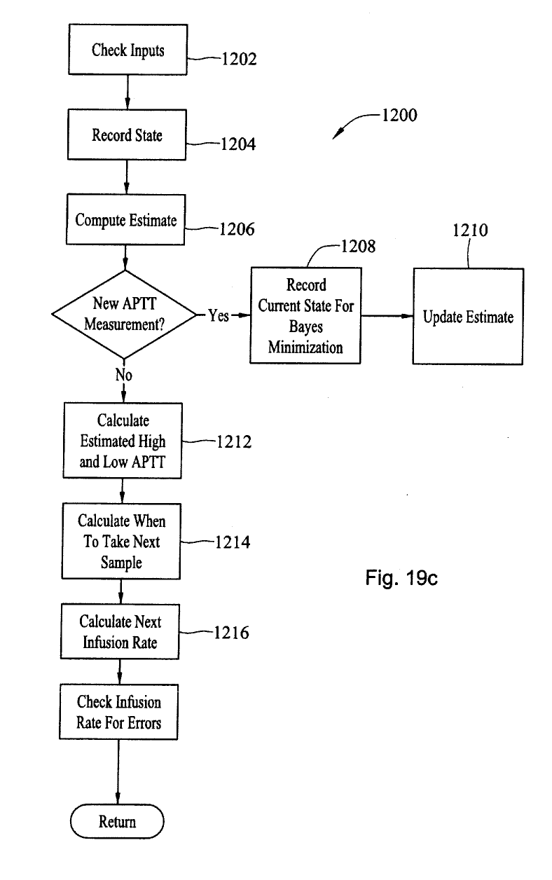

FIGS. 19a-c are flowcharts illustrating implementation of an embodiment of the control system including the pre-drug delivery phase and the drug delivery phase. FIG. 19a illustrates am implementation of a drug control system. First, in steps 1000 and 1002, the system acquires the patient information and protocol information such as the patient's baseline aPPT (based on a first aPPT measurement) and target aPPT. This information can be input by an operator and will be used to determine the initial condition for the patient response. In step 1004, the system the patient's aPPT measurement and in step 1006 the system acquires current heparin infusion rate. In step 1008 the system runs an algorithm to calculate the optimal heparin infusion rate for achieving the previously entered target aPPT rate. The algorithm is based on a pharmacodynamic model that is reiteratively adjusted to reflect the history of aPPT measurements and corresponding dose rates entered in steps 1004 and 1006 in order to tailor the model to reflect the patient's individualized heparin response. If the inputs are valid, the system transitions to either the pre-drug delivery phase 1100, the drug delivery phase 1200, or the post-drug clean up phase 1300.

In the pre-drug delivery phase 1100, illustrated in FIG. 19b, system is determines the initial condition of the patient response model based on information including whether the patient has been given a bolus in step 1102, whether the patient's measured aPPT is within normal range at step 1104 and whether the patient is on warfarin at step 1106. Once the patient's aPPT measurement is close to baseline, the initial parameters for the patient model can be set at step 1108 and the system can be transitioned into the drug delivery phase at steps 1110 or 1112 and the system transitions to a closed-loop system. As illustrated in step 1114, the system can remain in open loop mode wherein at step 1116, the infusion rate is halved every hour and until a valid aPPT measurement can be obtained.

In the drug delivery phase, illustrated in FIG. 19c, the system checks the inputs at step 1302, records the current state at step 1304 and estimates the parameters of the individual patient at step 1306 in order to adapt the model parameters to reflect the individualized estimated patient parameters. When a new aPPT measurement is available, the system uses Bayesian parameter estimation to estimates the parameters of the individual patient at step 1308 in order to update the estimate at step 1310 to adapt the model parameters to reflect the individualized estimated patient parameters. If a new aPPT measurement is not available, the system uses the current estimate computed at step 1306 to calculate the estimated high and low aPPT at step 1312. As step 1314, the uncertainty in the current estimate computed at step 1306 is used to calculate when to take the next blood sample for a new aPPT measurement. As step 1316, the protocol, including the updated pharmacodynamic model is used to calculate a new infusion rate. Referring back to the overall method illustrated in FIG. 19a, the system then uses the calculations made in the drug phase 1200 to set the next sample time in step 1010 and to set the new infusion rate in step 1012.

### Sampling System/Withdrawal Set.

The sampling system can be arranged to withdraw any biological fluid including blood, urine, interstitial fluid, or saliva. The preferred sample is blood. The sampling system preferably contains a bar code/RFID tag and interlock with the system to ensure patient safety and notify the medication control unit if any errors occur (e.g. occlusion, attempted removal, etc). The sampling system is capable of either intermittent sampling or could be adapted to continuous sampling based on the sensor(s).

The preferred embodiment of the sampling system incorporates an inflatable cuff 112 (blood pressure like cuff) and works in conjunction with the controller and sampler to ensure smooth withdrawal of blood. In one embodiment, two or more cuffs may be utilized. In the preferred embodiment of a multi-cuff system, one cuff 112 is located proximal of the point of insertion and the other cuff 114 is located distal to the point of insertion. The sampling system is coupled with a specific algorithm to inflate automatically prior to sampling (an automated corresponding to a tourniquet manually used for a lab blood draw) and use a sensing algorithm to set the pressure just above the systolic pressure to ensure a smooth draw and more frequent success to prevent vein collapse (especially in elderly).

The sampling system is preferably housed in a cassette that will fit into the device. In one aspect of the invention, an interlock system and optionally a bar code or RFID tag pair it with the IPMC.

In one system embodiment, the automated blood sampling system preferably comprises a tourniquet, an indwelling catheter, a pressure measuring system, a pump, a disposable set, an optical source and detector and a computer controlled adaptive algorithm. The tourniquet may be of any appropriate type, including hydraulic, pneumatic or mechanical, or any other fashion by which circumferential pressure can be applied to a limb. In one embodiment, the tourniquet optionally has a very low compliance, that is, it is relatively rigid system. Such a system has a relatively quick response time, with a fast on/fast off.

The tourniquet can be either above or below the point of insertion of the pressure monitoring catheter or system. If it is below the point of insertion, increased pressure may be utilized. The catheter may be “a single lumen catheter” or “a multi lumen catheter”. The pressure measuring system can be either invasive (via the indwelling catheter) or non-invasive (external pressure sensor). The catheter may be used to have a direct measure of venous pressure.

The pump may be of any type consistent with the application, such as a peristaltic pump, linear, rotary or cassette pump. A re-usable or disposable in-line transducer may be used to provide the pressure signal. If utilized, the disposable set interfaces with the pressure measuring system to provide real time or historic pressure measurement. Optionally, the pressure measuring system reads through the disposable set. In a preferred embodiment, pressure is measured transmurally, such as through use of an elastic segment of tubing laid across a strain gauge.

The optical sensor provides information to the adaptive algorithm. In the system, the presence of whole blood is indicated by absorbance of the optical signal, thus preventing it from reaching the optical detector. Optionally, the optical detector reads through the disposable set.

### The Multiple Tourniquet Embodiments

In one embodiment, multiple tourniquets are utilized adjacent the catheter. In the most preferred embodiment of this system, one tourniquet is disposed below the catheter and another is disposed above the catheter. Such a system provides the ability to meter the vessel dilation by adjusting each tourniquet pressure separately. While not limited to the following, various options for the pressure of the multiple cuffs are as follows:

in a first embodiment, applying pressure to cuff proximal to catheter,

in a second embodiment, applying pressure to cuff distal to catheter, keeping the pressure below the diastolic pressure,

in a third embodiment, for a distal location, use a pressure above diastolic pressure, or for a proximal approach uses a pressure above systolic pressure.

in a fourth embodiment, alternate between both cuffs, which can be used to induce venous distension and dilation.

By limiting pressure to just below diastolic (or just above or both) safety is increased as arterial flow is still permitted. The enhanced safety aspect of a tourniquet that operates near or below diastolic offers significant safety advantage (no pain, hemostasis, etc) and if operated in a narrow pressure band, the time to reach and/or adjust tour pressure is quite short, which is an advantage to ‘manipulate’ the vessel diameter somewhat.

As pressure in the vein drops, the pump rate (and therefore its vacuum) also drops to prevent vein collapse. As the pressure cuff enhances venous pressure, the pump speeds up. A goal is to maintain constant local venous pressure in the area of the catheter tip, most particularly proximal to the nearest valve in the vein. As venous pressure rises, so does the withdrawal rate of the pump. It may exceed baseline pressure (venous pressure with no external fluid moving in or out of the catheter) depending on the effect of tourniquet. Optionally, ramp rates may be varied.

This mechanically moves the catheter tip away from whatever might be blocking it by using reactionary force. If the infusion is fast, the catheter tip will have a force on it that moves it away from the valve or venous wall. Again, this might be in conjunction with the tourniquet manipulations.

### Systems and Methods for Enhancing Vein Lumen Diameter

In yet another embodiment, the algorithm alerts an infusion pump, fluidically connected to the indwelling catheter, to infuse saline or other fluid at a high rate. One effect of fast infusion is to enhance vein lumen diameter.

First, an infusion of saline may be used to enhance venous diameter. Optionally, this infusion may be used in conjunction with some tourniquet pressure.

Second, a local vasodilator may be used rather than saline if it does not interfere with the aPTT infusion, and is effective at dilating a vein. While saline may result in physical distention, other infusates have a dilating effect, e.g., nitroprusside, or other vasodilator known to those skilled in the art. Enhancers of nitrous oxide, delivered locally, may provide a vasodilatation effect. A very low concentration may be utilized. A fluid that produces very local vasodilation may be used to enhance sample withdrawal success rate.

Third, in one embodiment, the pressure to the tourniquet is oscillated. The oscillations may be rapid or slow. One advantageous result of the oscillations is to enhance venous dilation.

Fourth, a special multi-orifice catheter may be employed to avoided positional effects of the catheter opening.

### Infusion Systems and Methods

The algorithm may alert an infusion pump, fluidically connected to the indwelling catheter, to infuse saline or other fluid at a high rate to displace the catheter tip from the venous wall to enhance sample withdrawal.

Such an infusion results in mechanical movement of the catheter tip away from whatever might be blocking it by using a reactive force. Upon fast infusion the catheter tip will have a force on it that moves the catheter away from the valve, venous wall or other obstruction. Optionally, this technique may be used in conjunction with the tourniquet manipulations.

### Feedback Sensor(s)

The IPMC 110 is a modular system with the capability of providing feedback on different parameters from different medications or on more than one parameter (e.g., drug level, pharmacodynamic response) simultaneously. This is achieved by having the sensor be interchangeable in the device or by a sensor that can be used with more than one assay parameter. One embodiment, shown below in FIGS. 7A and 7B, is a cassette 160 which consists of multiple assays for different assays (e.g., a1 162, a2 164 (alternating); or a1 162, a2 164, a3 166, a4 168 (in sequence)). Thereby multiple assay parameters (e.g. aPTT, glucose concentration, potassium level) can be detected in sequence. The embodiment below preferably interlocks with the system and contains a barcode/RFID tag to ensure that the correct parameters are being measured.

In another aspect of the invention of the system, vital signs monitoring (e.g. ECG, blood pressure, Sp02) is integrated into the overall monitoring of the safety and state of patient. The blood pressure and heart rate can be analyzed using the cuff 112 that is part of the sampling system.

In one embodiment, a system for providing a set of individually sealed disposable cartridges for intermittently receiving and testing the biological fluid taken from the patient for intermittently or continuously monitoring one or more parameters such as activated partial thromboplastin time (aPTT) may be provided. In a first embodiment an assay device is contained in the original hermetically sealed pouch. The cartridge has 12 months (or more) of a shelf life. The pouch preferably is not optically clear, so the assay device is exposed to application of the sample and is then read by a reading device (including a source of light and sensor). In some embodiments, the assay device is device is exposed to application of the sample and is then read by a reading device (including a source of light and sensor) without removing the assay device from the pouch. Alternately, in some embodiments, the system includes a mechanical device that opens the pouch and removes an assay device from the pouch in order to expose the assay device to the application of the sample and read the assay device using a source of light and sensor).

In the second embodiment, the assay device is used without the original hermetically sealed pouch. The shelf life of a “naked” assay device, i.e., assay device removed from other packaging material, is on the order of a couple of hours. Typically, however, the series of assay devices is exposed to the Intensive Care Unit (ICU) environment for 2.5 days. Accordingly, it is desired to protect the “naked” assay devices for at least 3 days. An optically clear plastic cassette holds a set of “naked” assay devices. Each assay device is preferably placed in an individual nest. Preferably, each assay device is sealed by optically clear plastic foil (such as by ultrasonic techniques). The sample application procedure (puncture optically cleared plastic foil of one assay device) would expose only one assay device to ICU environment. The remaining assay devices would remain hermetically sealed until they are moved to a sample application site. Therefore, the last few assay devices will be sealed for more then 2 days.

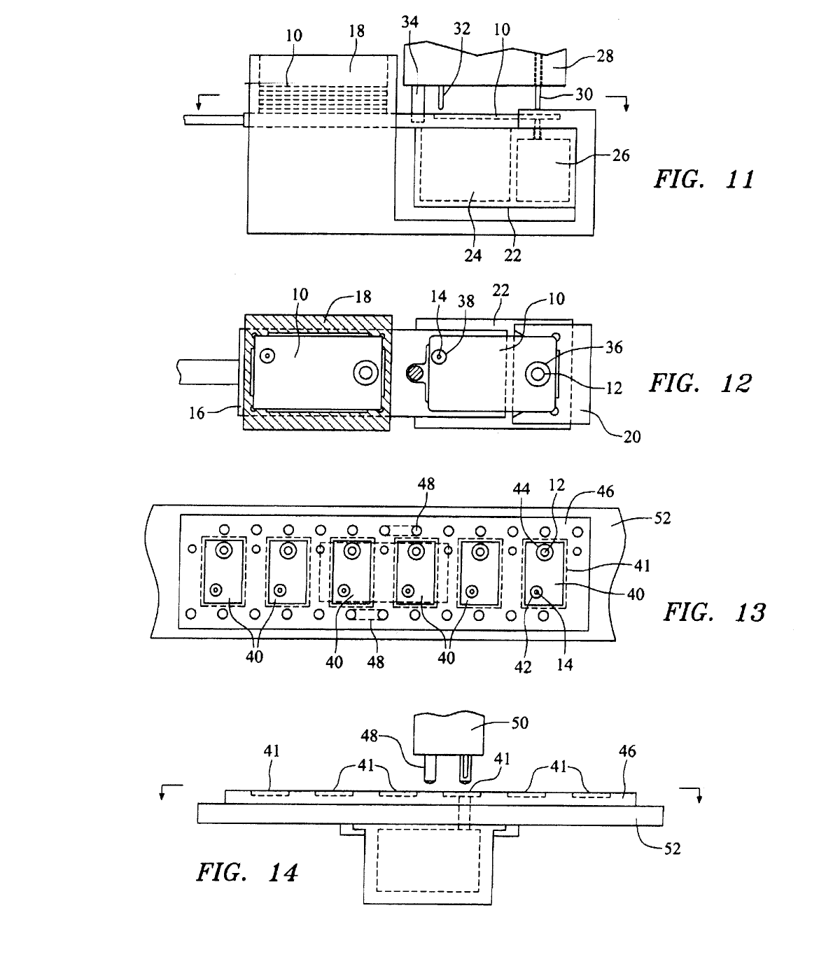

FIGS. 11 and 12 show side and top views, respectively, of a loading mechanism for an individual assay device, a sample application area, and waste reservoirs. The plurality of assay devices 10 are loaded in a stacked configuration in a stationary magazine 18. Each assay device has vent 14 and application site 12 hermetically sealed by sealant parts 36, 38, preferably made from thin film easily penetrated plastic material. A movable tray 16 pushes the assay device 10 one by one from the stationary magazine 18 into the sample application area defined by a preferably stationary guide-stopper 20. Needles 30 and 32 penetrate the seal 36 and 38, respectively, to open the vent 14 and to deliver a patient sample to the sample site 12 of the assay device 10. Alternately, the cartridges could be advanced “tractor feed” style to the sample area as illustrated in FIG. 18

The loaded assay device is then illuminated by a light source or other sensor and a measurement device, such as an aPTT measurement device measures an analyte in the fluid medium delivered to the assay device. Once the diagnostic reading has been performed, the discarded assay device 10 is striped from the movable tray 16 by the movable (e.g., up and down) pin 34 and falls down to a waste area 24 of a reservoir 22. The circuit flush fluid (containing, e.g., heparin, saline, and blood) is collected in waste reservoir 26. In some embodiments, the waste area 26 preferably contains materials to absorb the fluid from the used assay device

FIGS. 13 and 14 show side and top views, respectively, of slide tray arrangement for multiple tests site locations. The individual assay devices 40 are located in individual nest of the slide tray 46. Each individual assay device 40 has vent 43 and application 45 sites hermetically sealed by parts 42 and 44, respectively, made from thin film easily penetrated plastic material. The slide tray 46 is indexing on a top surface of a table 52 by indexing pins 48, mounted in an indexing mechanism 50.

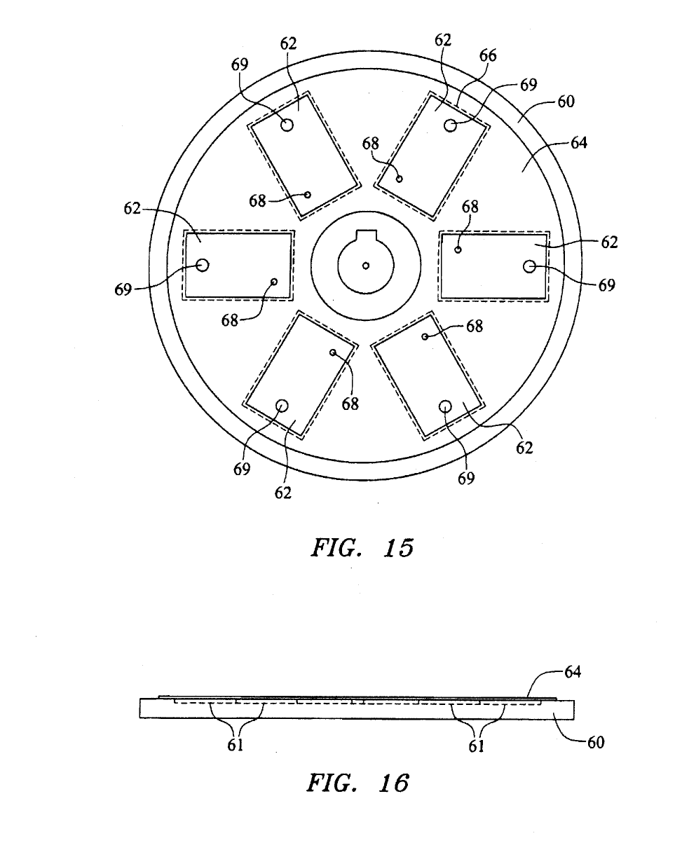

FIGS. 15 and 16 show top and side views, respectively, of a planar carousel arrangement handling multiple test site locations. The assay devices 62 are located in individual nest of the disc 60. The individual assay devices are covered by part 64 made from a thin film easily penetrated material. Each individual assay device 62 is hermetically sealed on the perimeter by welding part 64 to the disc 60. Welding line 66 is shown dashed in the figure. The vent 68 and application site 69 are automatically opened in each individual assay device 62 in the sample application area by puncturing the thin film 64.

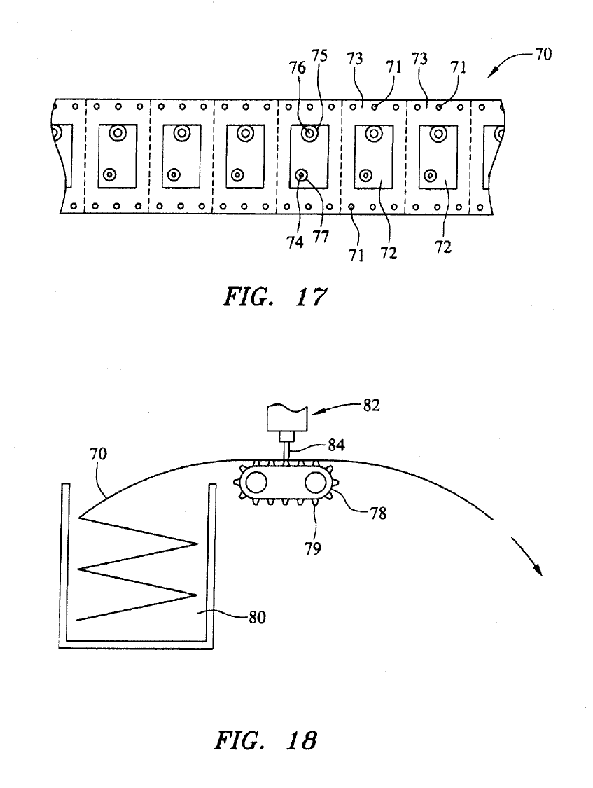

FIGS. 17 and 18 illustrate top and side views, respectively of a fan-fold arrangement 70 for multiple test site locations and a tractor-feed sample delivery mechanism 82. The disposable assay devices 72 are located in individual nests 73 of a continuous “tractor-feed,” fan-folded strip 70 that are each hermetically sealed by a thin film of easily penetrated plastic material. Each individual assay device 72 has vent 74 and sample application sites 76 hermetically sealed by parts 77 and 75, respectively, made from thin film easily penetrated plastic material. The fan-fold strip of disposable assays 70 are stored in a folded arrangement in a magazine 80 and sequentially advanced to the sample delivery mechanism 82 by a belt of indexing pins 79 mounted on an tractor feed indexing mechanism 78 that engage the perforated holes 71 located along the sides of the fan-fold strip 70 to advance an assay device 72 to a sample application area where needles 84 and 86 attached to the sample delivery device 82 will pierce the plastic film around parts 75 and 77 to expose the vent 74 and sample application site 76 and deliver a sample of a fluid to the assay device for intermittently performing a diagnostic measurement.

### Algorithm and Medication Control Unit (MCU)

The IPMC System is based on intermittent sampling or if the sensor allows, continuous measurement. It is important to note that the sampling system may take intermittent samples, and the MCU 130 uses algorithms to reconstruct patient's state, response and then calculate drug delivery rate based on intermittent samples. In addition, the optimal sampling time to take a sample can be determined by analyzing the response of the patient and if patient response is unexpected (e.g., in wrong direction) the medical delivery is halted and an alert or alarm is raised.

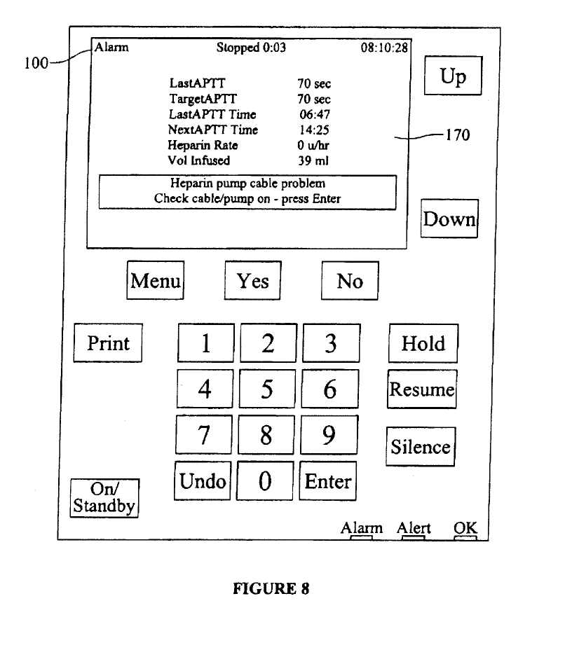

There is also an alarm/alert infrastructure/supervisory system 100 to oversee the entire MCU. If all aspects of the IPMC System are functioning there is a “green light” and delivery proceed. If there is an alert, (e.g., a non-critical problem that is potentially correctable) has been detected (e.g. sampling error, communication error, etc.) a yellow alert and audible alarm occurs. If a serious condition occurs (incorrect infusion rate, multiple missed samples, disconnected line) then the system immediately goes into alarm (red light, audible alarm, communication to central station). FIG. 8 shows a representative display of a monitor 170 for the system.

The adaptive algorithm controls the pneumatic or mechanical tourniquet to apply pressure or release pressure to the subject's extremity proximal (closest to the heart) to the indwelling catheter. In one implementation, the adaptive algorithm controls the tourniquet pressure based on real time and historic data both within patient and based on population data. The adaptive algorithm preferably adjusts the withdrawal rate of the pump based on real time and historical measurement provided by the pressure measuring system.

A heuristic algorithm is optionally included that ‘learns as it goes’ on a per-subject basis. Such a system preferably starts with a population basis.

Real-time venous pressure measurements may be included in the algorithm, if available. Alternatively, pressure may be measured indirectly, such as via external strain gauge.

The algorithm attempts to optimize the sample integrity, such as by maximizing the sample draw speed, to minimize sample time in the sample tube, to avoid sample degradation, e.g., degradation of aPTT measurements.

In yet another embodiment, the adaptive algorithm controls both the tourniquet pressure and the withdrawal rate based on real time and historic pressure data. The combination of these two ideally results in better sample draw than either factor individually. The adaptive algorithm may compensate for inferred venous pressure drop by altering the withdrawal rate. As pressure in the vein drops, the pump rate (and therefore its vacuum) also drops to prevent vein collapse. As the pressure cuff enhances venous pressure, the pump speeds up. The goal is to maintain constant local venous pressure in the area of the catheter tip and certainly proximal to the nearest valve in the vein. As venous pressure rises, so does the withdrawal rate of the pump, indeed, it may exceed baseline pressure (venous pressure with no external fluid moving in or out of the catheter) depending on the effect of tourniquet. Other variations may be utilized, such as ramp rates.

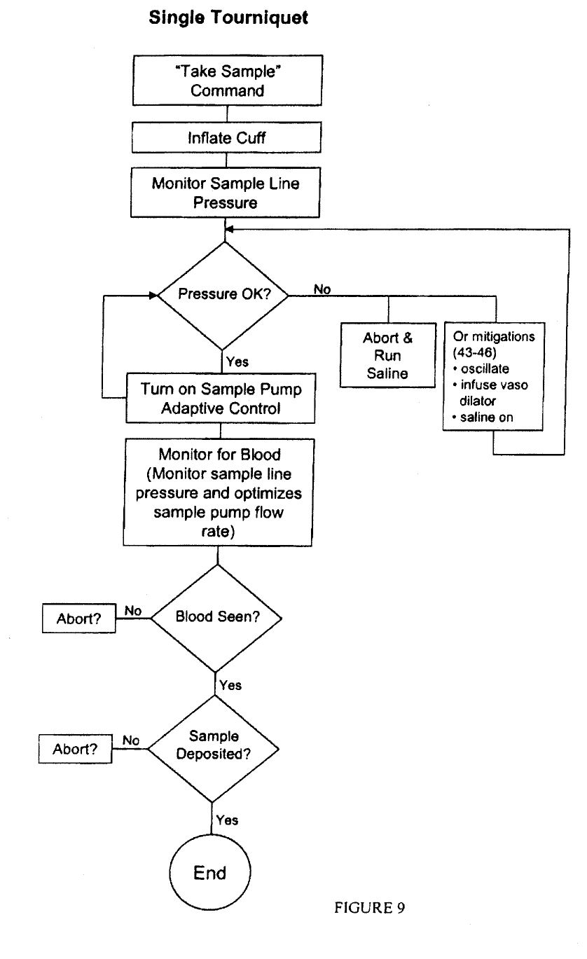

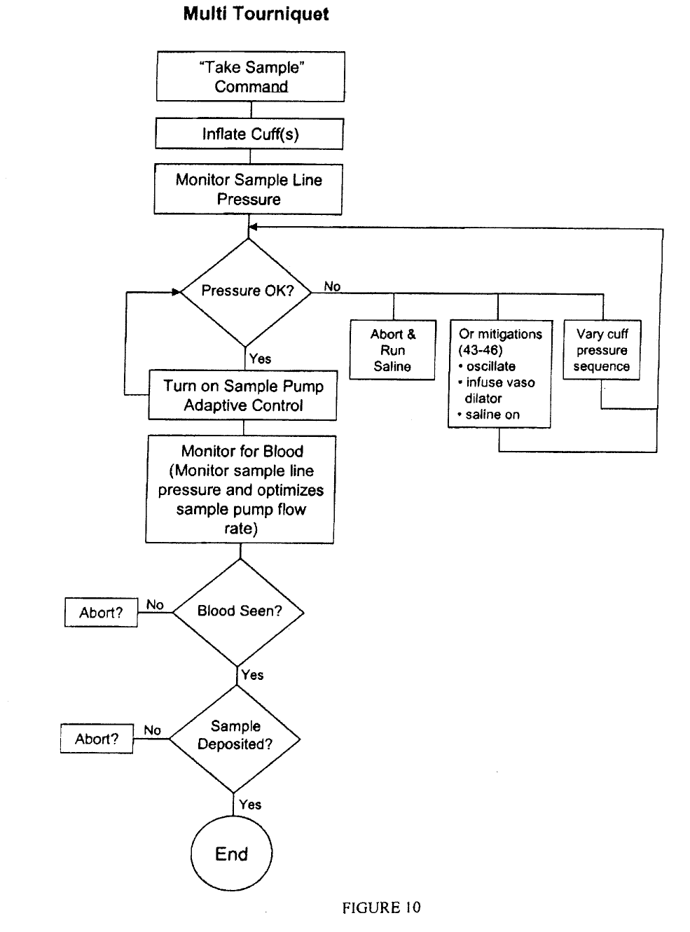

The adaptive algorithm may be implemented on a microprocessor or microcontroller. FIGS. 9 and 10 show flow charts for possible implementations of the systems and methods of the inventions. In FIG. 9, the system initially issues a “Take Sample” command. Next, the cuff is inflated. In the third step, the sample line pressure is monitored. If the pressure is within acceptable limits, the system proceeds to turn on the sample pump under adaptive control. At least while the pump is on, the system monitors for blood in the line. Preferably, the sample line pressure is also monitored, which is then used to optimize the sample pump flow rate. If no blood is seen, the sample is then deposited, and the system can then end. If blood is seen, an abort is an option. If (after step 3, above) the pressure is not within acceptable limits, the system any either (1) abort and run saline in the line, or (2) attempt various mitigation routines as discussed herein, including but not limited to oscillation of the pressure, infusion of a vaso dilator, or to turn the saline on.

The process of the multi-tourniquet system is as described for FIG. 9, but further includes the option after the third step in the event the pressure is not within acceptable limits, to vary the cuff pressure sequence. Possible sequences could include, but are not limited to, inflate the proximal cuff, recheck the pressure, and if it is not within acceptable limits, to inflate the distal cuff, and deflate the proximal cuff. If the pressure is still not within acceptable limits, the distal cuff could be deflated and the procedure repeated. These sequences may be performed in any order or combination or permutation.

### System and Method Control

In one embodiment, the tourniquet pressure is limited to approximately or slightly lower than diastolic pressure to prevent hemostasis in the extremity.

By limiting pressure to just below diastolic (or just above or both) we are increasing safety as arterial flow is still permitted. The enhanced safety aspect of a tourniquet that operates near or below diastolic offers significant safety advantage (no pain, hemostasis, etc) and if we operate in a narrow pressure band the time to reach and/or adjust tour pressure may be quite short. This can advantageously serve to ‘manipulate’ the vessel diameter.

### Adaptive Drug Delivery Control System

In some embodiments, the IPMC System operates as a closed-loop drug delivery system uses patient response and rule based decision making methods to achieve operator specified responses for therapeutic purposes. In the preferred embodiment, the IPMC system delivers heparin based on optimally sampled aPTT measurements to achieve a desired anticoagulation status of the patient. The overall drug delivery system consists of a hardware system and an expert, rule-based, control system. In one embodiment, the system, apparatus and methods operate in an automatic feedback controlled manner to achieve drug delivery. Stated otherwise, the system operates to monitor, sample, determine time and dosing requirements, and cause dosing without intervention by health care professionals (save for example a required response to an alert or alarm condition). For example, the sampling system 110 may take intermittent samples, the sensor/assay 120 may then perform a diagnostic analysis on the sample and the MCU 130 uses algorithms to reconstruct patient's state, response and then calculate drug delivery rate based on analysis of the intermittent samples. In addition, the optimal sampling time to take a sample can be determined by analyzing the response of the patient and if patient response is unexpected (e.g., in wrong direction) the medical delivery is halted and an alert or alarm is raised.

In an alternate embodiment, the hardware can also be replaced in whole or in part by a user that provides, for example, the results (aPTT) at the times requested by the system, and manually adjusts the infusion rate in response to the expert-rule based control system. In this system the rule-based control system, the system is flexible enough to accept inputs (e.g., aPTT) at times other than that requested, and adapt to changes in infusion rate other than recommended by the system. In this mode, the system functions much like a GPS navigator for an automobile, where a driver makes a wrong turn, and it recalculates the route to get to the desired destination (albeit using taking a longer time to reach the destination).

In one embodiment, the hardware system, such as the blood sampling set/withdrawal set 110 (or user) acquires a patient venous blood sample that is assayed to determine the anticoagulation status of the patient as determined by the analytically measured aPTT value. The MCU 130 further receives operator specified information and based on the inputs, outputs the desired rate of heparin drug infusion that is then used to determine an infusion rate. The method generally consists of determining the heparin infusion based on a target aPTT value input by the operator. The target value may change during the course of the treatment.

More particularly, the method generally comprises the following steps:
Determine the initial condition of the patient based on a first aPTT measurement. Since it is unknown whether heparin has already been administered to the patient, either as a bolus or a continuous infusion, the system will determine the starting point of the response based on the results of the first measurement.
If the patient has received a heparin bolus, it is likely that the first measurements will be out of range. In this case, the control option is to stay in an open-loop mode until a valid measurement can be obtained. In the open-loop mode, a steady rate infusion is administered calculated to achieve the target setting for an average responder and a new measurement is scheduled at the next hour.
If the patient has not received previous heparin and the baseline aPTT is within normal range, the initial condition for the patient response shall be set at the value of the first measured aPTT value. In all other cases, the population based estimate for the baseline aPTT shall be used as the reference baseline patient response.
Determine the initial condition of the patient response model based on the operator entered information. This information shall contain the weight of the patient, the sex of the patient, the age of the patient, the smoker status of the patient, status on whether the patient has received heparin, status whether patient is on warfarin medication and status whether patient is on other thrombolytic medications.
Determine the target aPTT target based on operator entered information.
The transition into closed-loop drug delivery is triggered when a valid measurement is obtained, a valid aPTT target is set and no alarms are active. The infusion rate is set to achieve the target in the bolus case. For the baseline case, the infusion rate is set to geometrically achieve the target rate.
If automated, the infusion can be interrupted if an alarm is triggered in the system or the operator chooses to stop the infusion. In this case the control system shall continue to monitor the patient state without administrating drug. If the operator chooses to restart the infusion, and the alarm status is clear, the control system shall resume the infusion to achieve the target selection.
The target aPTT can be changed by the operator during the treatment. In this case, the control system shall adjust the infusion to reach the newly selected target value.

An important aspect of the drug delivery system is the adaptive feature to adjust the properties of the dynamic system based on the knowledge from newly acquired measurements. The basis for the control system is a parameterized representation of the average patient response to heparin. Since humans show a wide variability in drug response, the parameters for the dynamic system should be adjusted to more accurately describe the measurement of the particular patient response. For the heparin application, the dynamic system includes multiple parameters and a non-linear input-output response. Different numerical methods exist to find the optimal parameter set to satisfy a least-square error criterion between the model response and the measurement set.

Another important aspect of the drug delivery system is the determination of the optimal sampling time. Since the process of acquiring a patient sample and conducting the analytical measurement may be inconvenient to patient and or caregiver and impose additional cost, it is imperative that the measurement are taken judiciously to satisfy the requirements of the therapeutic goal while minimizing other safety or treatment concerns. Since the dynamic system has a known patient variability, one can determine the expected variability of the patient response. If these estimates exceed a specified threshold, the control system may determine an infusion rate that is suboptimal for the treatment and mandate a new measurement. New measurements will improve not only the confidence in the patient status but will also update the parameters of the dynamic system thereby reducing the variability of the predicted patient response. The result of these stochastic analyses is a control system that learns quickly about the patient through initial frequent measurements and achieves a tight control response through the improvements in specific patient response. However, control system should also be able to adapt to data provided at times other than requested, and infusion rates other than those recommended and still obtain the desired endpoint, as long as the specified infusion rate is not out of range, or will result in an undesirable endpoint, at which point the system should provide an alarm or alert to the user and cease recommendations until the conditions are modified.

### Details of the Control System

The determination of a dynamic model is the foundation for the description of the input-output behavior and the core of the control system.

### Model for Assumption of Linear Elimination

The aPTT response to heparin infusion is described using a model structure in which heparin infusion produces an aPTT elevation above a baseline value. The change in the logarithm of the aPTT is proportional to the heparin concentration. A one compartment pharmacokinetic model has been frequently employed to describe the relationship between the heparin concentration and the infusion rate in hemodialysis applications. For a linear model, the time rate of change of the compartmental concentration H, is

$$\frac{d H}{d t} = - {k}_{10} H ( t ) + \frac{u ( t )}{{V}_{d}} \qquad ( 1 )$$

where u is the heparin infusion rate, k₁₀ is the elimination rate constant, and V_{d} is the apparent volume of distribution, which corresponds to the blood volume for heparin. The aPTT response, R, to heparin infusion is the magnitude of the elevation of the log(Aptt) above the logarithm of the baseline value, log 10(Aptt_{base})

R=log 10(Aptt)−log 10(Aptt_{base}) (2)

which may be expressed as

Aptt=10^{R} Aptt_{base} (3)

log 10(Aptt)=R+log 10(Aptt_{base}) (4)

Since the response is proportional to the heparin concentration,

R=mH (5)

the time rate of change of the response may be written as

$$\frac{d R}{d t} = - {k}_{10} R + Su ( t ) \\ where \\ S = \frac{m}{{V}_{d}} . \qquad ( 6 )$$

### Model for Assumption of Nonlinear Elimination

For heparin, the rate of elimination is reduced at high doses. This nonlinear elimination is thought to be due to the effect of a saturable mechanism of elimination in reticuloendothelial and endothelial cells acting in parallel with a linear renal elimination. For a model of heparin pharmacokinetics having a linear and a saturable mechanism the time rate of change of the concentration is:

$$\frac{d H}{d t} = - [ {k}_{l} + \frac{{V}_{m}}{{K}_{m} + H ( t )} ] H ( t ) + \frac{u ( t )}{{V}_{d}} . \qquad {( 7 )}^{20}$$

For this nonlinear model, since R=mH, the time rate of change of the response may be written as

$$\frac{d R}{d t} = - [ {k}_{l} + \frac{{V}_{m 1}}{{K}_{m 1} + R ( t )} ] R ( t ) + Su ( t ) \\ where \\ S = \frac{m}{{V}_{d}} . \qquad ( 8 )$$

Mungall²¹ assumed heparin elimination was governed only by Michalis-Menten kinetics, such that the heparin concentration is given by

$$\frac{d H}{d t} = - [ \frac{1}{{V}_{d}} \cdot \frac{{V}_{m}}{{K}_{m} + H ( t )} ] H ( t ) + \frac{u ( t )}{{V}_{d}} \qquad ( 9 )$$

and the response is given by

$$\frac{d R}{d t} = - [ \frac{1}{{V}_{d}} \cdot \frac{{mV}_{m}}{{mK}_{m} + R} ] R + \frac{m}{{V}_{d}} u ( t ) . \qquad ( 10 )$$

For this model, the parameterization

θ=[mVₘKₘV_{d}Aptt_{base}] (11)

may be employed, where

$$\begin{matrix}cov ( θ ) = \begin{matrix}E [ {θθ}^{T} ] & ( 12 )\end{matrix} \\ = E [ \begin{matrix}{m}^{2} & {mV}_{m} & {mK}_{m} & {V}_{d} m & {mAptt}_{base} \\ {mV}_{m} & {V}_{m}^{2} & {K}_{m} {V}_{m} & {V}_{d} {V}_{m} & {V}_{m} {Aptt}_{base} \\ {mK}_{m} & {K}_{m} {V}_{m} & {K}_{m}^{2} & {V}_{d} {K}_{m} & {K}_{m} {Aptt}_{base} \\ {V}_{d} m & {V}_{d} {V}_{m} & {V}_{d} {K}_{m} & {V}_{d}^{2} & {V}_{d} {Aptt}_{base} \\ {mAptt}_{base} & {V}_{m} {Aptt}_{base} & {K}_{m} {Aptt}_{base} & {V}_{d} {Aptt}_{base} & {Aptt}_{base}^{2}\end{matrix} ]\end{matrix}$$

Of course, the alternative parameterization θ=[SVₘKₘAptt_{base}] could be employed.

Alternatively, the model (7) may be written as

$$\frac{d R}{d t} = - [ L + \frac{V \cdot K}{K + R ( t )} ] R ( t ) + Su ( t ) \qquad ( 13 )$$

so that when R(t)<<K

$$\frac{d R}{d t} = - [ L + V ] R ( t ) + Su ( t )$$

and when R(t)>>K

$$\frac{d R}{d t} = - L \cdot R ( t ) - V \cdot K + Su ( t )$$

For this model, the parameterization

θ=[LSVKAptt_{base}]^{T} (14)

is employed, where

$$\begin{matrix}cov ( θ ) = E [ {θθ}^{T} ] \\ = E [ \begin{matrix}{L}^{2} & LS & LV & LK & {LAptt}_{base} \\ LS & {S}^{2} & SV & SK & {SAptt}_{base} \\ LV & SV & {V}^{2} & VK & {VAptt}_{base} \\ LK & SK & VK & {K}^{2} & {KAptt}_{base} \\ {LAptt}_{base} & {SAptt}_{base} & {VAptt}_{base} & {KAptt}_{base} & {Aptt}_{base}^{2}\end{matrix} ]\end{matrix} \qquad ( 15 )$$

For the model described by Mungall²², most of the population parameters are listed in the reference.

### Discrete Time Model

For computation, the model might be cast into a discrete-time state-space form in which the system matrices vary with time to account for the nonlinear elimination.

R(t+tᵢₙₜ)=A(t)·R(t)+B(t)·U(t) (16)

Note that A and B would vary with time in (16) to approximately account for the nonlinear elimination.

Alternatively, the equation of the model (13) can be solved symbolically over one time interval, tᵢₙₜ, to yield an approximate solution for R(t+tᵢₙₜ)

$$R ( t + {t}_{int} ) = \frac{1}{2 a} \cdot ( - b + {( {b}^{2} - 4 ac )}^{1 / 2} ) \\ where \\ a = {t}_{int} \cdot L + 1 \\ c = - K \cdot R ( t ) + {t}_{int} \cdot S \cdot K \cdot U ( t ) \\ b = \frac{c}{K} + {t}_{int} \cdot V \cdot K + {t}_{int} \cdot L \cdot K + K . \qquad ( 17 )$$

If the model structure in (9) is used, then L=0 and a=1.

Equations (3) and (4) presented earlier describe the Aptt and log(Aptt).

Aptt=10^{R} Aptt_{base} (3)

log 10(Aptt)=R+log 10(Aptt_{base}). (4)

The measured system output, y(t), may be taken as log 10(Aptt). If the model is accurate, it is assumed that the measurement error v(t), in the output, y(t), is given by

v(t)=y(t)−h(z(t))) (18)

where

h(z(t))=R+log 10(Aptt_{base}) (19)

is the predicted system output. If the error in the measurement of the Aptt is a percentage, of the Aptt,

The standard deviation of the measurement error in the Aptt is assumed to be a percentage (say 5 percent) of a patient's actual Aptt. The variance of the error in the measured log 10(Aptt) is approximated by

$${σ}_{{y}_{j}}^{2} = {[ \frac{log ( 1 + w )}{log ( 10 )} ]}^{2}$$

where w is the percentage error in the measured Aptt.

In (19) the model (4) is described using the variables

x(t)=R(t) (20)

θ=[log(L)log(S)log(V)log(K)log(Aptt_{base})]^{T} (21)

(where log indicates natural logarithm) and the state vector is

$$z ( t ) = [ \begin{matrix}x ( t ) \\ θ ( t )\end{matrix} ] . \qquad ( 22 )$$

The state equation is then written as

$$\begin{matrix}z ( t + {t}_{int} ) = f ( z ( t ) , u ( t ) ) + [ \begin{matrix}v ( t ) \\ 0\end{matrix} ] . & ( 23 ) \\ f ( z ( t ) , u ( t ) ) = [ \begin{matrix}\frac{1}{2 a} \cdot ( - b + {( {b}^{2} - 4 ac )}^{1 / 2} \\ θ\end{matrix} ] & ( 24 )\end{matrix}$$

Alternatively,

h(z(t))=x(t)+log 10(Aptt_{base}) (25)

may be written

$$h ( z ( t ) ) = [ \begin{matrix}1 & 0 & 0 & 0 & 0 & \frac{1}{log ( 10 )}\end{matrix} ] \cdot z ( t ) \qquad ( 26 )$$

(note log(10)=2.3025851) such that

$$y ( t ) = [ \begin{matrix}1 & 0 & 0 & 0 & 0 & \frac{1}{log ( 10 )}\end{matrix} ] \cdot z ( t ) + v ( t ) . \qquad ( 27 )$$

### Adaptive Control

Control law. A control law is used to compute the heparin infusion rate that would be required to move the aPTT from the aPTT value predicted using the model at discrete time t to the set point over the discrete time interval from t to t+tᵢₙₜ. The predicted and desired Aptt are logarithmically transformed to compute the response R. The value Rₜ is the target response value that the next infusion rate will be computed to achieve. For the model (16)

$$U ( t ) = \frac{{R}_{t} ( t + {t}_{int} ) - A ( t ) \cdot R ( t )}{B ( t )} . \qquad ( 28 )$$

For the model (17), the model equation may be solved symbolically to yield an approximate control law

U(t)=[a·Rₜ²(t+tᵢₙₜ)+(b₁−R(t))·Rₜ(t+tᵢₙₜ)−K·R(t)]/tᵢₙₜ/S/(K+Rₜ(t+tᵢₙₜ)) (29)

with

b₁=tᵢₙₜ·V·K+tᵢₙₜ·L·K+K.

The solutions (17) and (29) determined symbolically appear to be accurate as long as R(t+tᵢₙₜ) is close to R(t) in (17) and as long as Rₜ(t+tᵢₙₜ) is close to R(t) in (29).

The infusion rate is constrained by the limitations of the infusion device and by the fact that the infusion rate cannot be negative. Thus, the set point will not always be achieved in one discrete time interval with the constrained infusion rate. Infusion pumps are generally capable of adjusting the volumetric infusion rate in fixed increments of typically 1 ml/hr over a range from 0 to a maximum pumping rate which is typically 1000 ml/hr. The new aPTT achieved using the constrained infusion rate is predicted for time t+tᵢₙₜ using the model, and the new aPTT prediction is used in computing the next infusion rate for the interval from t+tᵢₙₜ to t+2tᵢₙₜ.

Parameter estimation. When an aPTT measurement is available, the parameters of the individual patient are estimated and the model parameters are adapted to those of the patient. Bayesian parameter estimation is employed because it is a powerful method that takes into account both the model prediction and its variability based upon the population pharmacokinetics, and the measured aPTT and the variability of the measurement process. Parameters may be estimated by iterative minimization of a Bayesian objective function (such as equation (30) below) or parameters may be estimated recursively using the extended Kalman Filter system (EKF) below. Iterative minimization provides accurate parameter estimates, but is time consuming because in each iteration, a model must be used to repeatedly predict the system output using the parameter estimate for the iteration. Recursive parameter estimation is not as accurate, but may be adequate and may serve better in initial demonstrations because the fast execution would facilitate more interactive simulation.

If the correlation between two parameters in the patient population is significant, then theoretically, knowing one parameter would infer some knowledge about the other. A Bayesian objective function that is appropriate for the case in which there is no correlation between the parameters in the patient population is

$${Bayes}_{obj} = {\sum }_{i = 1}^{{n}_{θ}} \frac{{( \overline{log {θ}_{i}} - \hat{log {θ}_{i}} )}^{2}}{{σ}_{log {θ}_{i}}^{2}} + {\sum }_{j = 1}^{{n}_{meas}} \frac{{( {y}_{j} - h ( {z}_{j} ) )}^{2}}{{σ}_{{y}_{j}}^{2}} . \qquad ( 30 )$$

In (30), l̅o̅g̅ θ̅ᵢ̅ is one component of the vector of the means of the natural logarithms of the population parameters,

l̅o̅g̅ θ̅=[ l̅o̅g̅(̅L̅)̅ l̅o̅g̅(̅S̅)̅ l̅o̅g̅(̅V̅)̅ l̅o̅g̅(̅K̅)̅ l̅o̅g̅(̅A̅p̅t̅t̅_̅{̅b̅a̅s̅e̅}̅)̅]^{T}

where the variance of the natural logarithm of a parameter is σ_{log θ}_{ᵢ}². Note that

$${σ}_{log {θ}_{i}}^{2} = log ( 1 + \frac{{σ}_{{θ}_{i}}^{2}}{{\overline{θ}}_{i}^{2}} )$$

A Bayesian objective function that takes into account correlation between the parameters in the patient population would have the form

$${Bayesc}_{obj} = {( \overline{log {θ}_{i}} - \hat{log {θ}_{i}} )}^{T} {V}^{- 1} ( \overline{log {θ}_{i}} - \hat{log {θ}_{i}} ) + {v}^{T} {N}^{- 1} v \qquad ( 31 )$$

where V is the covariance matrix of the population parameters and N is the covariance matrix of the measurement errors, v. N is assumed to be of a diagonal structure with σ_{y}_{ⱼ}² as the diagonal elements. Thus, if V reflected no correlation between the parameters then its off-diagonal terms would be zero and (31) would be equivalent to (30).

After parameter estimation, the parameter vector used to predict the patient response is updated, the Aptt response prediction at the sample time is revised to take into account the new measurement using an extended Kalman filter state estimator, and the Aptt prediction is recomputed over the time period from the sample time to the current time based on the new parameter estimates and the revised response prediction of the Aptt at the sample time.

### Forecasting System

The forecasting system is based on an extended Kalman filter (EKF) structured for combined parameter and state estimation. Between measurements, the uncertainty in the state estimate is propagated from the time of the last measurement, m, to the current time, t, through the time update part of the EKF as the covariance matrix is updated. When a measurement is available at the time of the next measurement, n, the state and the covariance matrix are updated in the measurement update. The covariance of the system output is computed based on the covariance of the state and parameters; the square root of that covariance is used as a confidence interval for the system output. The EKF is used to propagate the uncertainty in the state estimate.

The forecast of the confidence interval of the model output at time t based on the last measurement at time m is given by

S₁=H(θ,\hat{x}(t|m))P(t|m)H(θ,\hat{x}(t|m))^{T} (32)

where P(t|m) is the covariance matrix for z(t) and

$$\begin{matrix}H ( θ , \hat{x} ( t \vert m ) ) = \frac{\partial }{\partial z} {( h ( z ( t \vert m ) ) \vert}_{θ = \hat{θ}} \\ = [ \begin{matrix}1 & 0 & 0 & 0 & 0 & \frac{1}{log ( 10 )}\end{matrix} ] .\end{matrix} \qquad ( 33 )$$

### Sampling System

The forecasted confidence interval is used in an system that determines the sampling schedule by selecting sampling times that prevent the forecasted confidence interval from exceeding a threshold. The confidence interval is computed into the future. The next sample is scheduled for a time that allows the measurement for that sample to be entered into the system and used in adaptive control before the threshold is exceeded. Consider an example where the delay between sampling and the availability of a measurement for adaptive control is 5 minutes, and the forecasting system computes that the threshold will be exceeded at 243 minutes. The sampling time would be 238 minutes.

### Details of the Supervisory System

The supervisory system provides additional safeguards to ensure patient is not put at risk due to operator or equipment errors. These controls check the inputs and outputs of the system.

Patients with a bolus and an initial aPTT in excess of 150 seconds, receive an infusion that targets an “average” patient to reach a target level halfway between baseline and selected target. This rate is halved every 90 minutes until a valid aPTT (<150 s) is achieved. The system will alert the operator if the patient's aPTT exceeds the range for a prolonged period of time.

The system keeps an estimate of the patient's aPTT based on the dynamic model and the previous measurements. The estimate consists of an expected value within a normal range (standard deviation). If new measurements deviate from the estimate by more than 2 standard deviations, the supervisor system will alert the operator and request a new measurement.

The supervisor methods monitor the outputs of the feedback system to make sure all boundary conditions of infusion rate and infusion duration are within expected range.

The supervisor methods monitor the operator input with the first measured patient response to ensure that the initial conditions are consistent with the entered patient profile.

### Medication Delivery Technology

The medication delivery technology optionally consists of intravenous infusion pumps 142, syringe pumps, implantable pumps, transdermal iontophoretic systems. The preferred embodiment is an intravenous infusion pump. The preferred delivery route is intravenous, but other portals such as intrarterial, transdermal, peritoneal, subcutaneous, or buccal could also be used.

In the preferred embodiment, the pump is an integral part of the system rather than connected by an interface. This prevents any potential safety issues including 1) communication errors between devices, 2) incorrect information being sent between devices, 3) loss of control of device, 4) undetected error that is missed by pump and not detected by the medication control unit. Optionally, the system, will contain a bar code reader 150 that can read the identity of the medication being delivered as well as its concentration, and patient for whom it is intended.

### Alerts and Alarms

Optionally, a safety algorithm alerts the caregiver that a sample can not be obtained unless a set of predefined conditions are met. Various alerts and alarms may be used. A clinical alert can also be incorporated to notify a clinician that drug is scheduled to be delivered, and require approval by the physician (directly or through a remote connection) before administration.

### Applications

The systems and methods described herein may be used for automated blood sampling, and then used in combination with other systems, methods and applications. Of particular utility are closed-loop systems which use the described automated blood sampling in combination with a diagnostic assay to provide an analysis of the blood, and where that analysis is used in providing a drug or other material to the patient. Most preferably, the closed-loop system is fully automated from the blood sampling, to the diagnostic assay, to the provision of drug delivery.

### Additional Aspects

The system preferably includes telemetry (either wired via ethernet or like, or wireless like bluetooth or WIFI) to communicate information to central station. The system has the ability to pair the system with the patient's instructions to make sure the right patient is being started on the right drug.

While various embodiments have been described herein, they may be used in combination with multiple embodiments. The embodiments may be combined in order to optimize successful sampling and control.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

### REFERENCES

¹The Joint Commission Sentinel Event Alert: Preventing errors relating to commonly used anticoagulants Issue 41, Sep. 24, 2008.
²Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996; 93:870-878.
³Cheng S, Morrow D A, Sloan S, Antman E M, Sabatine M S. Predictors of initial nontherapeutic anticoagulation with unfractionated heparin in ST-segment elevation myocardial infarction. Circulation. 2009 Mar. 10; 119(9):1195-202. Epub 2009 Feb. 23.
⁴Anand et al. Relationship of Activated Partial Thromboplastin Time to Coronary Events and Bleeding in Patients with Acute Coronary Syndrome Who Receive Heparin. Circulation. 2003; 107:2884-2888.
⁵Cannon et al. Automated Heparin Delivery System to Control Activated Partial Thromboplastin Time. Circulation. 1999; 99:751-756.
⁶Alchemia's generic fondaparinux a potential beneficiary of heparin product recall. Alchemia Ltd. press release: Mar. 27, 2008. <http://www.alchemia.com>
⁷IMS National Sales Perspective Report. IMS Health Inc. June 2008.
⁸Ibid.
⁹MEDMARX® is a national database that tracks and trends adverse drug reactions and medication errors.
¹⁰C. Peterson, C. Ham, T. Vanderveen. Improving Heparin Safety: A Multidisciplinary Invited Conference. Hospital Pharmacy, Vol. 43, No. 6, pp 491-497.
¹¹Ibid.
¹²Granger C B, Hirsh J, Califf R M et al. for the GUSTO-I Investigators. Activated partial thromboplastin time and outcome after thrombolytic therapy for acute myocardial infarction: results from the GUSTO-I Trial. Circulation. 1996; 93:870-878.
¹³Anand et al. Relationship of Activated Partial Thromboplastin Time to Coronary Events and Bleeding in Patients with Acute Coronary Syndrome Who Receive Heparin. Circulation. 2003; 107:2884-2888.
¹⁴Ibid.
¹⁵T. K. Gandhi et al. Protocols for High-Risk Drugs: Reducing Adverse Drug Events Related to Anticoagulants. Agency for Healthcare Research and Quality (AHRQ).
¹⁶T Y Wang, E Peterson, M Ohman et al. Excess Heparin Dosing Among Fibrinolytic-treated Patients with ST-Segment Elevation Myocardial Infarction. American Journal of Medicine (2008) 121:805-810.
¹⁷The Joint Commission Sentinel Event Alert: Preventing errors relating to commonly used anticoagulants Issue 41, Sep. 24, 2008.
¹⁸C. Peterson, C. Ham, T. Vanderveen. Improving Heparin Safety: A Multidisciplinary Invited Conference. Hospital Pharmacy, Vol. 43, No. 6, pp 491-497.
¹⁹Smart Pumps Are Not Smart On Their Own. Institute for Safe Medication Practices Newsletter, Apr. 19, 2007.
²⁰Cannon, Christopher P., et al., “Automated Heparin-Delivery System to Control Activated Partial Thromboplastin Time, Circulation, 1999; 751-756 at 752.
²¹Kershaw, Beverly, White, Richard H., Mungall, Dennis, et al., “Computer-Assisted Dosing of Heparin”, Arch. Intern. Med. Vol. 15-t., May 9, 1994, 1005-1010, at 1007.

What is claimed:

1. A system for determining a diagnostic result from a fluid medium comprising:
a series of assay devices,
a measurement device to provide diagnostic results,
a storage device for said assay devices,
an advancement mechanism said assay devices through a sample application area, and
a mechanism for dispensing a fluid medium to an assay device.

2. The system of claim 1, further comprising an integrated waste area for collecting waste.

3. The system of claim 2, wherein the integrated waste area contains materials to absorb a liquid component of the waste.

4. The system of claim 1 wherein the set of assay devices comprise individual cartridges hermetically sealed inside of individual aluminum foiled pouches.

5. The system of claim 4, wherein the assay devices are integrated into a continuous strip.

6. The system of claim 1 wherein the measurement device comprises a source and/or sensor to measure an analyte in the fluid medium.

7. The system of claim 1 wherein the storage device for said assay devices comprises a framed structure for placement of said assay devices.

8. The system of claim 4 wherein the storage device further includes an optical reader of said assay device bar code.

9. The system of claim 1 wherein the advancement mechanism for said assay devices comprises an actuator that advances said assay device from storage device through a sample application area.

10. The system of claim 6 wherein the actuator is an electromechanical actuator.

11. The system of claim 6 wherein the actuator is a pneumatic actuator.

12. The system of claim 1 further comprising a mechanism for exposing an optical reading site on an assay device to a source of light and optical reader and the application site to a sample dispensing device, the mechanism comprising a mechanical device(s) that opens a moisture impermeable pouch and removes an assay device from said pouch.

13. The system of claim 12, wherein the mechanism for exposing an optical reading site on an assay device contained within a moisture impermeable pouch comprises aligning the optical reading site to the source of light by puncturing a seal in the pouch and exposing the optical reading site without removing the assay device from the pouch.

14. The system of claim 12 wherein the mechanism of exposing an optical reading site on an assay device contained within a moisture impermeable pouch to the source of light and optical reader and application site to a sample dispensing device comprises at least one mechanical device that opens the moisture impermeable pouch in said areas.

15. The system of claim 1, further comprising a removal mechanism for removing the assay device from the application area after completion of one diagnostic reading.

16. The system of claim 15 wherein the mechanism of removing said assay device from application area after completion of one diagnostic reading consists of an electromechanical or pneumatic device that deposits the cartridge to a waste reservoir.

17. A system for storing assay devices used to measure a diagnostic result in a fluid medium comprising:
a series of assay devices;
a storage device for said assay devices; and
an integrated waste area that contains materials to absorb a liquid component of a waste material.

18. A system for determining activated partial thromboplastin time (aPTT) in a fluid medium employing a device comprising
a cassette containing a set of assay devices,
an aPTT measurement device,
an advancement mechanism for advancing said assay devices through a sample application area, and
a reservoir for collecting a liquid waste.

19. The system of claim 18, wherein the cassette contains a set of assay devices consisting of an optically clear support structure with individual nests for each individual said assay device.

20. The system of claim 19, wherein each assay device is hermetically sealed in own individual nest with optically clear plastic material.

21. The system of claim 19, wherein the support structure includes a drum.

22. The system of claim 19 wherein the support structure includes a rack.

23. The system of claim 17, wherein the advancement mechanism for the assay device consist of rotating mechanism delivering said assay devices to a sample application area.

24. The system of claim 23, wherein the rotating mechanism is a drum.

25. The system of claim 17 wherein the advancement mechanism for the assay devices consist of indexing mechanism delivering said assay devices to a sample application area.

26. The system of claim 25 wherein the indexing mechanism is a rack.

27. A system for determining Prothrombin time (PT/INR) in a fluid medium employing a device comprising:
a cassette containing a set of assay devices;
a PT/INR measurement device;
an advancement mechanism for advancing said assay device to a sample application area; and
a reservoir for collecting a liquid waste.

28. A method for determining the infusion rate for delivering heparin to a patient comprising the steps of:
(a) obtaining a patient blood sample;
(b) measuring the patient's Activated Partial Thromboplastin Time (aPTT);
(c) inputting the patient aPTT measurement into a processor;
(d) inputting an aPTT target for the patient into the processor; and
(e) using the processor to calculate a heparin infusion rate for the patient to achieve the target aPTT, the processor implementing a protocol including a dynamic patient model based on a pharmacodynamic model of heparin response that utilizes:
(i) the patient's past history of infusion rates and
(ii) the current infusion rate
to calculate the heparin infusion rate.

29. The method of claim 28, further comprising reiteratively repeating steps (a)-(e) at selected intervals of time, wherein the dynamic patient model is adjusted to reflect the patient's individualized heparin response.

30. The method of claim 29, wherein at least one parameter in the dynamic patient model is adjusted using Bayesian estimation to take into account the patient's aPTT measurements.

31. The method of claim 29, wherein time interval for repeating steps (a)-(e) is predefined by the processor according to pharmacodynamic model of heparin response.

32. The method of claim 29, the time interval is reiteratively calculated after each repetition based on the adjusted dynamic patient model to take into account patient's response to the current infusion rate.

33. The method of claim 29, wherein the protocol further includes a forecasting model for determining the confidence interval in the current estimated patient response and wherein the confidence interval is used to calculate the time interval for repeating steps (a)-(e).

34. The method of claim 28, further comprising adjusting the parameters of the dynamic patient model to reflect the patient's individualized heparin response based on the patient's measured aPTT and the current infusion rate.

35. The method of claim 28, wherein the dynamic patient model includes multiple parameters and wherein the protocol provides a non-linear input-output response.

36. The method of claim 28, wherein the dynamic patient model is updated after each heparin infusion to reflect patient's individualized heparin response based on the patient's measured aPTT and the current infusion rate.

37. The method of claim 28 further comprising:
calculating the optimal sampling time interval for re-measuring the patient's aPTT.

38. The method of claim 28, further comprising triggering an alert/alarm in response to certain preset conditions.

39. The method of claim 38, wherein the preset conditions are selected from the following group consisting of: when the patient test results are out of range for specified infusion rate, when the processor has not received sample input for certain period of time

40. The method of claim 38, wherein the alarm stops the delivery of heparin.

41. The method of claim 28, further comprising
initiating heparin delivery to a patient at a rate calculated by the processor;
monitoring the patient response to the heparin delivery, wherein the monitoring comprises taking a blood sample form the patient and measuring the patient's aPTT according to a sampling frequency determined by the processor;
adjusting the dynamic patient model to reflect the patient's individualized heparin response;
using the processor to calculate an updated heparin infusion rate based the revised protocol; and
adjusting the heparin delivery to the updated infusion rate.

42. A method for determining the sampling schedule for controlling the heparin delivery rate to a patient to maintain an optimal heparin delivery rate comprising the steps of:
(a) obtaining a patient blood sample;
(b) measuring the patient's Activated Partial Thromboplastin Time (aPTT);
(c) inputting the patient aPTT measurement into a processor;
(d) inputting an aPTT target for the patient into the processor; and
(e) using the processor to calculate a time interval for re-measuring the patient's aPTT to maintain an optimal infusion rate for the patient, the processor implementing a protocol including a dynamic patient model based on a pharmacodynamic model of heparin response that utilizes:
(i) the patient's past history of infusion rates and
(ii) the current infusion rate
to calculate the optimal time interval for re-measuring the patient's aPTT.

43. The method of claim 42, further comprising repeating steps (a)-(e), wherein the dynamic patient model is adjusted to reflect the patient's individualized heparin response.

44. The method of claim 43, wherein adjusting the dynamic patient model comprises adjusting at least one parameter in the dynamic patient model using Bayesian estimation to take into account the patient's aPTT measurements.

45. The method of claim 42, wherein the processor further includes a forecasting model for determining the confidence interval in a current estimated patient response and wherein the processor utilizes the confidence interval to calculate the time interval for the re-measuring the patient's aPTT.

46. The method of claim 45, further comprising the step of inputting a maximum threshold for the confidence interval into the processor.

47. The method of claim 46, wherein the processor utilizes the threshold to calculate the time interval for re-measuring the patient's aPTT.

48. The method of claim 45, further comprising reiteratively repeating steps (a)-(e) wherein the confidence interval is re-calculated after each patient aPTT measurement.
